(19) United States
(12) Patent Application Publication
(10) Pub. No.: US 2021/0369812 A1
(43) Pub. Date: Dec. 2, 2021

(54) OSTEOINDUCTIVE PEPTIDES, COMPOSITIONS, IMPLANTS, AND METHODS OF USE

(71) Applicant: The Board of Regents of the University of Oklahoma, Norman, OK (US)

(72) Inventors: Chuanbin Mao, Norman, OK (US); Kegan Sunderland, Blaine, ME (US)

(21) Appl. No.: 16/647,242

(22) PCT Filed: Apr. 9, 2019
(86) PCT No.: PCT/US2019/026546

§ 371 Date: Mar. 13, 2020

Related U.S. Application Data
(60) Provisional application No. 62/655,340, filed on Apr. 10, 2018.

(51) Int. Cl. A61K 38/18; A61K 47/69; A61K 47/02; A61K 9/00; A61K 35/28; A61P 19/08; A61L 27/56; A61L 27/38; A61L 27/48; A61L 27/46; A61L 27/34; A61L 27/06

(52) U.S. Cl. CPC A61K 38/1875; A61K 47/6929; A61K 47/02; A61K 9/0024; A61K 35/28; A61P 19/08; A61L 2400/12; A61L 27/3834; A61L 27/48; A61L 27/46; A61L 27/34; A61L 27/06; A61L 2430/02; A61L 27/56

(57) ABSTRACT

Osteoinductive, bone morphogenic protein receptor-binding peptides are disclosed. The peptides may be used to coat or infuse scaffolds for use as implants into bone for enhancing the growth, proliferation, and differentiation of mesenchymal stem cells and/or osteoblasts in the bone.

### CROSS-REFERENCE TO RELATED APPLICATIONS

The present application claims priority to U.S. Provisional Application Ser. No. 62/655,340, filed Apr. 10, 2018, the entirety of which is hereby expressly incorporated herein by reference.

### BACKGROUND

There is a pressing need for bone regenerative materials for repair of defects due to, for example, traumatic injuries, skeletal deformities, removal of bone tumors, non-unions (broken bone failing to heal), avascular necrosis, and osteoporotic fractures. However, repairing large bone defects in load-bearing sites poses a major challenge as replacement implants must have the appropriate mechanical strength, biocompatibility, shape, and size. Producing replacement implants by 3D printing is a versatile approach to addressing these problems because implants can be customized to fit any patient-specific complex defect within the resolution capabilities of 3D printing. When the implants are constructed as porous scaffolds with microscale features, they can be seeded with human mesenchymal stem cells (hMSCs), the microscale features can serve to enhance nutrient transport and help induce vascularization. However, there remains a need for improving the cell growth and proliferation and vascularization of such cell-seeded structures after implantation. It is to addressing such a need that the present disclosure is directed.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The figures are not necessarily to scale and certain features and certain views of the figures may be shown as exaggerated in scale or in schematic in the interest of clarity and conciseness. The patent or application file contains at least one drawing executed in color. Copies of this patent or patent application publication with color drawing(s) will be provided by the Office upon request and payment of the necessary fee.

FIG. 1 shows results of an evaluation of comparative ELISA testing of the BMPR-binding peptides (SEQ ID NOS: 1-5, 8, 9) in binding to BMPR1A (A) or BMPR2 (B). Groups having the same lowercase letter assigned (above the bar) were not statistically different from each other. Groups having different letters assigned (above the bar) were statistically different from each other. The ELISA (n=4) was carried out using a phage concentration of 2×10¹² phage/ml which displays the peptide indicated on the p3 coat protein of M13 phage. The primary antibody was mouse anti-g3p (p8) IgG. The secondary antibody was anti-mouse IgG conjugated with horseradish peroxidase. The substrate, 3,3′,5,5′-Tetramethylbenzidine was detected at 450 nm after stopping the reaction with 1 M HCl.

FIG. 2 shows results of hMSC cell growth and proliferation on phage films as measured by the percent reduction of alamar blue. hMSCs were grown for 47 hours (A) or 71 hours (B) before incubating with alamar blue and measuring the absorbance at 570 nm and 600 nm. The percent reduction of alamar blue was then calculated (higher percent reduction=more growth). Groups (bars) sharing any of the same letters above them are not significantly different. Groups having all different letters are significantly different.

FIG. 3 shows microphotographs of immunofluorescence representing production of osteogenic protein markers osteopontin (OPN), osteocalcin (OCN), and collagen type 1A (COL). OPN was detected with the primary antibody for OPN (RbpAB to osteopontin ab8448 from Abcam). The secondary antibody used was Goat Anti-Rabbit IgG H&L (Alexa Fluor 555) (ab150078) from Abcam (red). OCN was detected with the primary antibody for OCN [OC4-30] (ab13418) from Abcam). The secondary antibody used was Goat Anti-Mouse IgG H&L (Dylight 650) preadsorbed from abcam ab96882 (red). COL was detected using a primary antibody for COL (RbpAb to collagen 1 ab34710). The secondary antibody used was Goat Anti-Rabbit IgG H&L (Alexa Fluor 555) (ab150078) from abcam (red). Cell nuclei were stained by DAPI (blue) for all samples, and actin was stained by phalloidin (green). The positive control was osteogenic media from Lonza ((hMSC Osteogenic BulletKit). All other samples were grown in basal media (MSCGM BulletKit (PT-3238 & PT-4105) from Lonza. Peptides were displayed on M13 phages and developed into thin films for which the hMSCs were grown on for 26 days.

FIG. 4 shows Osteopontin expression by hMSCs after 26 days of culturing on phage films displaying the indicated peptides or controls. Gene expression was normalized to the reference gene, GAPDH. All fold changes are relative to a negative control (basal media with no peptides or phage) which represents the baseline at zero-fold change. The sequence VHVPLHRGAVSA (SEQ ID NO:2) causes a fold-change greater osteopontin production than the Lonza osteogenic media, the positive control. The sequence SGTQDSMVGWNK (SEQ ID NO:3) induced greater osteopontin production than the wild type, but not greater than the Lonza osteogenic media. All data represent the mean±standard deviation. (*P≤0.05, **P≤0.01, ***P≤0.001).

FIG. 5 shows Collagen type1A expression (a control that should be the same for all samples) of hMSCs after 26 days of culturing on phage films displaying the indicated peptide or controls. Gene expression was normalized to the reference gene, GAPDH. All fold changes are relative to a negative control (basal media with no peptides or phage) which represents the baseline at zero-fold change. No significant difference was observed between the conditions chosen. All data represent the mean±standard deviation. (*P≤0.05, **P≤0.01, ***P≤0.001).

FIG. 6 shows Osteocalcin expression of hMSCs after 26 days of culturing on phage films displaying the indicated peptide or controls. Gene expression was normalized to the reference gene, GAPDH. All fold changes are relative to a negative control (basal media with no peptides or phage) which represents the baseline at zero-fold change. All data represent the mean±standard deviation. (*P≤0.05, **P≤0.01, ***P≤0.001).

FIG. 7 shows M13 phage scaffolds arranged through freeze casting and imaged by SEM. Pure M13 phage scaffolds at different magnifications (A and B). M13 phages combined with collagen and hydroxyapatite nanorods at different magnifications (C and D). M13 phages mixed with collagen and hydroxyapatite which were cross-linked using EDC.

FIG. 8 shows freeze-casted HAP. (A) Center top view of scaffold; (B) Edge top view of scaffold; (C) zoomed in view of lamellar structure; (D) high magnification view of a chasm of the lamellar structure.

FIG. 9 shows freeze casted HAP which has been sintered. (A) Center top view; (B-D) top view at increasing magnification. The lamellar structure can be seen.

FIG. 10 shows 3D printed titanium alloy scaffolds. (A) Camera image of scaffolds. (b SEM top view of scaffold. (C) SEM zoomed in view of a pore on the surface of a titanium scaffold. (D) SEM zoomed in view of a pore and the surrounding region coated in phage fibers through double-sided freeze casting. (E) SEM high magnification of phage fibers aligned with the freezing direction on titanium scaffold. (F) SEM-EDS mapping of titanium (red) and carbon (green) at the same location as (D). (G) EDS map spectrum for (F).

FIG. 11 shows 3D printed scaffolds seeded with cells and phage. (A) SEM side view image of cells on titanium scaffold. (B) SEM-EDS mapping of carbon (green) which shows the location of the cells on the side of a titanium scaffold. (C) Map sum spectrum for image B. (D) SEM image of cells and phage together on a titanium scaffold. (E) Carbon mapping of image D showing the carbon in the cells and phages. (F) Map sum spectrum for image E.

FIG. 12 shows Micro-CT images of scaffolds implanted in a rat radial bone defect model. Images were taken 4 weeks after the defect was created. Increased healing was best observed in VHVPLHRGAVSA alone and a combination of TAKYLPMRPGPL+RDYHPRDHTATW.

FIG. 13 shows Micro-CT images of scaffolds implanted in a rat radial bone defect model. Images were taken 12 weeks after the defect was created. Increased healing was best observed in several peptide coated samples.

FIG. 14 shows a top down view of a section of each cylindrical-shaped implant condition stained with HE. The dark black grids are the titanium and the cells can be seen in purple/pink. The scaffolds supported the penetration and growth of cells even to the deepest regions of the scaffolds. These implants were imaged after 4 weeks of implantation into the radial defect model.

FIG. 15 shows Von Kossa stains of 4-week implants in rat radial defect bone models viewed from the top down of the cylindrical scaffolds. The dark black grids are titanium. Other black areas are heavy calcium deposits. Gray areas are lighter calcium deposits. The lightly colored pink/purple are cells. New bone tissue was observed to be best around the scaffolds labeled VHVPLHRGAVSA and the combined TAKYLPMRPGPL+RDYHPRDHTATW.

### DETAILED DESCRIPTION

A phage display technique called biopanning was used to select peptides capable of binding bone morphogenic protein receptors (BMPRs) present on the surface of hMSCs. In nature, bone morphogenic proteins (BMPs) bind to these BMPRs, inducing hMSCs to differentiate into bone regenerative osteoblast cells. BMPs are part of the transforming growth factor-β superfamily and function as key regulators of cellular growth, differentiation, and tissue formation. While recombinant BMPs can be used to induce osteoblast differentiation, they are expensive to produce. As disclosed herein, we discovered several peptides, including but not limited to, six peptides having the amino acid sequences TAKYLPMRPGPL (SEQ ID NO:1), VHVPLHRGAVSA (SEQ ID NO:2), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), which bind to the bone morphogenic protein receptors, BMPR1A and BMPR2. These peptides of the present disclosure, among others, induce MSCs (e.g., hMSCs) to differentiate into osteoblasts. The peptides can be linked to (i.e., coated onto) implant materials (e.g., scaffolds) or films, such that the resulting peptide-implant compositions induce osteogenic activity and osteoinductive activity. The peptides may be linked to the surface of the implants or films via linkers, or via phage viruses that express and bear the peptides. Implantable scaffolds can be coated with these osteogenic peptides for, for example, enhancing bone regeneration and osteoblast growth in the scaffold in subjects treated for major bone defects. Other types of implants such as plates and screws used in bone repair or dentistry (or other scaffolds, implants, structures, hydrogels, pastes, or compositions) can be used as well. These peptides can be efficiently displayed on a human-safe virus (e.g., M13 phage), producing hundreds to thousands of copies of a peptide per phage (and titers around, e.g., 10¹³ phages per amplification of the virus), making the production and use of these peptides highly cost effective. The peptides of the present disclosure can be used for replacing antibody detection/imaging systems for the BMPRs as well as replacing expensive protein-based osteoblast differentiation strategies.

Before further describing various embodiments of the compositions and methods of the present disclosure in more detail by way of exemplary description, examples, and results, it is to be understood that the embodiments of the present disclosure are not limited in application to the details of methods and compositions as set forth in the following description. The embodiments of the compositions, structures, and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the compositions, structures, and methods of the present disclosure have been described in terms of particular embodiments, it will be apparent to those of skill in the art that variations may be applied to the compositions, structures, and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein. A list of abbreviations used herein is supplied in Table 1.

TABLE 1
Abbreviations

| | |
|---|---|
| AAV | Adeno-associated viruses |
| ALP | Alkaline phosphatase |
| ABTS | Azino-bis(3-ethylbenzothiazole sulfonic acid) |
| | diammonium salt |
| Phage | Bacteriophages |
| BMP | Bone morphogenic protein |
| BMPR1A | Bone morphogenic protein receptor type 1A |
| BMPR2 | Bone morphogenic protein receptor type 2 |
| BSA | Bovine serum albumin |
| Xgal | 5-Bromo-4-chloro-3-indolyl-β-D-galactoside |
| CRISPR/Cas | Clusters of regularly interspaced short |
| | palindromic repeat and associated |
| | genes system |
| COL | Collagen type 1A |
| CAD | Computer-aided design |
| CCMV | Cowpea chlorotic mottle virus |
| CPMV | Cowpea mosaic virus |
| DNA | Deoxyribonucleic acid |
| dNTP | Deoxynucleoside triphosphate |
| DMSO | Dimethyl sulfoxide |
| DTT | Dithiothreitol |
| ddH2O | Double distilled water |
| dsDNA | Double-stranded deoxyribonucleic acid |
| EDS | Energy-dispersive X-ray spectroscopy |
| EDC | 1-ethyl-3-(3-dimethylaminopropyl) carbodiimide |
| | hydrochloride |
| EDTA | Ethylenediaminetetraacetic acid |
| ECM | Extracellular matrix |
| HCl | Hydrochloric acid |
| HAP | Hydroxyapatite |
| hMSCs | Human mesenchymal stem cells |
| IPTG | Isopropyl-β-D-thiogalactoside |
| LB | Luria broth |
| mRNA | Messenger ribonucleic acid |
| 1D | One-dimensional |
| OCN | Osteocalcin |
| OPN | Osteopontin |
| PBS | Phosphate buffered saline |
| PCR | Polymerase chain reaction |
| qPCR | Quantitative polymerase chain reaction |
| PEG | Polyethylene glycol-8000 |
| RNA | Ribonucleic acid |
| SEM | Scanning electron microscopy |
| SLM | Selective laser melting |
| ssDNA | Single-stranded deoxyribonucleic acid |
| NaCl | Sodium chloride |
| STL | Stereo Lithography |
| TMV | Tobacco mosaic virus |
| TBS | Tris-buffered saline |
| TBST | Tris-buffered saline + 0.1% v/v Tween-20 |
| TEM | Transmission electron microscopy |
| 2D | Two-dimensional |
| UV | Ultraviolet |

All patents, provisional applications, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein, including for example, U.S. Provisional Application Ser. No. 62/655,340.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. Any two values within the above ranges, e.g., 88 and 444 therefore can be used to set the lower and upper boundaries of a range (e.g., 88-444) in accordance with the embodiments of the present disclosure.

The term “pharmaceutically acceptable” refers to compounds, compositions, and structures which are suitable for administration to humans and/or animals without undue adverse side effects such as toxicity, irritation and/or allergic response commensurate with a reasonable benefit/risk ratio.

By “biologically active” is meant the ability to modify the physiological system of an organism without reference to how the active agent has its physiological effects.

As used herein, “pure,” “substantially pure,” or “isolated” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein the term “high specificity” refers to a specificity of at least 90%, or at least 91%, or at least 92%, or at least 93%, or at least 94%, or at least 95%, or at least 96%, or at least 97%, or at least 98%, or at least 99%. Where used herein the term “high sensitivity” refers to a sensitivity of at least 90%, or at least 91%, or at least 92%, or at least 93%, or at least 94%, or at least 95%, or at least 96%, or at least 97%, or at least 98%, or at least 99%.

The terms “subject” and “patient” are used interchangeably herein and will be understood to refer to a warm blooded animal, particularly a mammal or bird. Non-limiting examples of animals within the scope and meaning of this term include dogs, cats, rats, mice, guinea pigs, horses, goats, cattle, sheep, zoo animals, Old and New World monkeys, non-human primates, and humans. The methods, kits, and systems encompassed herein are therapeutic for treating bone wounds and defects in such subjects and patients.

“Treatment” refers to therapeutic treatments. “Prevention” refers to prophylactic treatment measures to stop a condition from occurring. The term “treating” refers to administering the composition to a patient for therapeutic purposes, and may result in an amelioration of the condition or disease.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent (e.g., the peptides disclosed herein) which is sufficient to exhibit a detectable biochemical and/or therapeutic effect, for example without excessive adverse side effects (such as toxicity, irritation and allergic response) commensurate with a reasonable benefit/risk ratio when used in the manner of the inventive concepts. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

The following abbreviations may be used herein for amino acids: alanine:ala:A; arginine:arg:R; asparagine:asn:N; aspartic acid:asp:D; cysteine:cys:C; glutamic acid:glu:E; glutamine:gln:Q; glycine:gly:G; histidine:his:H; isoleucine:ile:I; leucine:leu:L; lysine:lys:K; methionine:met:M; phenylalanine:phe:F; proline:pro:P; serine:ser:S; threonine:thr:T; tryptophan:trp:W; tyrosine:tyr:Y; and valine:val:V.

The term “analog” means a chemically related form of that amino acid having a different configuration, for example, an isomer, or a D-configuration rather than an L-configuration, or an organic molecule with the approximate size and shape of the amino acid, or an amino acid with modification to the atoms that are involved in the peptide bond, to confer resistance to peptidases and proteases to a peptide. The phrases “amino acid” and “amino acid sequence” include one or more components which are amino acid derivatives and/or amino acid analogs comprising part or the entirety of the residues for any one or more of the 20 naturally occurring amino acids indicated by that sequence. For example in an amino acid sequence having one or more tyrosine residues, a portion of one or more of those residues can be substituted with homotyrosine.

The term “peptide” is used herein to designate a series of amino acid residues, connected one to the other typically by peptide bonds between the alpha-amino and carbonyl groups of the adjacent amino acids to form an amino acid sequence. In certain embodiments, the peptides can range in length from 2 to 10 to 15 to 25 to 40 to 60 to 75 to 100 amino acids, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids. The term “polypeptide” or “protein” is used herein to designate a series of amino acid residues, connected one to the other typically by peptide bonds between the alpha-amino and carbonyl groups of the adjacent amino acids, wherein the length is longer than a single peptide. A peptide conjugate refers, in a non-limiting embodiment, to a compound comprising a peptide of the present disclosure which is conjugated (e.g., covalently linked, directly or indirectly via a linker sequence) to another molecule, such as a carrier molecule such as a protein or other polymeric molecule, e.g., a polyethylene glycol (PEG) molecule, or other therapeutic compound such as a drug, or an imaging or diagnostic moiety and wherein the peptide retains its activity (e.g., binding, targeting, imaging, etc) even when conjugated to the molecule. A “fusion protein” or “fusion polypeptide” refers to proteins or polypeptides (and may be used interchangeably) which have been created by recombinant or synthetic methods to combine peptides in a serial configuration. The peptides of the present disclosure may be produced using any nucleotide sequence which encodes the desired amino acid sequence. Any of the peptides described herein or active variants thereof may be used to make the peptide conjugates of the present disclosure.

Peptides of the present disclosure and the nucleic acids which encode them include peptide and nucleic acid variants which comprise substitutions (conservative or non-conservative) of the native amino acids or bases. For example, the peptide variants include, but are not limited to, variants that are not exactly the same as the sequences disclosed herein, but which have, in addition to the substitutions explicitly described for various sequences listed herein, additional substitutions of amino acid residues (conservative or non-conservative) which substantially do not impair the activity or properties of the variants described herein.

Amino acids are classified into “groups” having chemically similar structures and chemical properties. The term “hydrophobic” amino acid means a group of aliphatic amino acids alanine (A or ala), glycine (G or gly), isoleucine (I or ile), leucine (L or leu), proline (P or pro), and valine (V or val), the terms in parentheses being the one letter and three letter standard code abbreviations for each amino acid, and a group of aromatic amino acids includes tryptophan (W or trp), phenylalanine (F or phe), and tyrosine (Y or tyr). These amino acids confer hydrophobicity as a function of the length of aliphatic and size of aromatic side chains in an amino acid sequence. The term “hydrophilic” amino acid means a group of amino acids including arginine (R or arg), asparagine (N or asn); aspartic acid (D or asp), glutamine acid (E or glu); glutamine (Q or gln), histidine (H, his), lysine (K or lys), serine (S or ser), and threonine (T or thr).

The term “neutral” amino acid means a group of amino acids including alanine (A or ala), asparagine (N or asn), cysteine (C or cys), glutamine (Q or gin), glycine (G or gly), isoleucine (I or ile), leucine (L or leu), methione (M or met), phenylalanine (F or phe), proline (P or pro), serine (S or ser), threonine (Tor thr), tyrosine (Y or tyr), tryptophan (W or tip), and valine (V or val). The term “acidic” amino acid means a group of amino acids including aspartic acid (D or asp) and glutamic acid (E or glu). The term “basic” amino acids includes a group including arginine (R or arg), histidine (H or his), and lysine (K or lys).

The term “charged” amino acid means a group of amino acids including aspartic acid (D or asp), glutamic acid (E or glu), histidine (H or his), arginine (R or arg) and lysine (K or lys), which confer a positive (his, lys, and arg) or a negative (asp, glu) charge at physiological values of pH in aqueous solutions of peptides. The term “polar” amino acid means a group of amino acids including arginine (R or arg), lysine (K or lys), aspartic acid (D or asp), glutamine acid (E or glu), asparagine (N or asn), and glutamine (Q or gln).

One of ordinary skill in the art would readily know how to make, identify, select or test such variants for receptor targeting activity against the same receptors targeted by the native peptides. Particular examples of conservative amino acid substitutions include, but are not limited to, gly:ala substitutions; val:ile:leu substitutions; asn:glu:his substitutions; asp:glu substitutions; ser:thr:met substitutions; lys:arg:his substitutions; and phe:tyr:trp substitutions. Other types of substitutions, variations, additions, deletions and derivatives that result in functional variant peptides are also encompassed by the present disclosure, and one of skill in the art would readily know how to make, identify, or select such variants or derivatives, and how to test for receptor binding activity of those variants.

Further examples of conservative amino acid substitutions include, but are not limited to, ala to gly, ser, or thr; arg to gln, his, or lys; asn to asp, gln, his, lys, ser, or thr; asp to asn or glu; cys to ser; gln to arg, asn, glu, his, lys, or met; glu to asp, gln, or lys; gly to pro or ala; his to arg, asn, gln, or tyr; ile to leu, met, or val; leu to ile, met, phe, or val; lys to arg, asn, gln, or glu; met to gln, ile, leu, or val; phe to leu, met, trp, or tyr; ser to ala, asn, met, or thr; thr to ala, asn, ser, or met; trp to phe or tyr; tyr to his, phe or trp; and val to ile, leu, or met. Conservative substitutions include amino acid substitution of one or more amino acid residues with another amino acid from the same group. Peptides include one or more amino acid deletions or amino acid additions of amino acids internally or to the N-terminus and C-terminus.

The term “homologous” or “% identity” as used herein means a nucleic acid (or fragment thereof) or a peptide having a degree of homology to the corresponding natural reference nucleic acid or peptide that may be in excess of 60%, or in excess of 65%, or in excess of 70%, or in excess of 75%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%, or other specific percentages described herein. For example, in regard to peptides, the percentage of homology or identity as described herein is typically calculated as the percentage of amino acid residues found in the smaller of the two sequences which align with identical amino acid residues in the sequence being compared, when four gaps per 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In at least one embodiment “% identity” represents the number of amino acids or nucleotides which are identical at corresponding positions in two sequences of a peptide or nucleic acids encoding similar peptides. For example, two amino acid sequences each having 12 residues will have at least 75% identity when at least 9 of the amino acids at corresponding positions are the same, at least 83% identity when at least 10 of the amino acids at corresponding positions are the same, or at least 92% identity when at least 11 of the amino acids at corresponding positions are the same.

Where a sequence is described herein as having “at least X % identity to” a reference sequence, this is intended to include, unless indicated otherwise, all percentages greater than X %, such as for example, (X+1)%, (X+2)%, (X+3)%, (X+4)%, and so on, up to 100%.

The terms “polynucleotide sequence” or “nucleic acid,” as used herein, include any polynucleotide sequence which encodes a peptide product including polynucleotides in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded DNA may be the coding strand, also known as the sense strand, or it may be the non-coding strand, also referred to as the anti-sense strand. The peptide may be expressed using polynucleotide sequence(s) which differ in codon usage due to the degeneracies of the genetic code or allelic variations.

The terms “infection,” “transduction,” and “transfection” are used interchangeably herein and mean introduction of a gene, nucleic acid, or polynucleotide sequence into cells such that the encoded peptide or protein is expressed. The polynucleotides which encode peptides or proteins of the present disclosure may comprise additional sequences, such as additional coding sequences within the same transcription unit, controlling elements such as promoters, ribosome binding sites, transcription terminators, polyadenylation sites, additional transcription units under control of the same or different promoters, sequences that permit cloning, expression, homologous recombination, and transformation of a host cell, and any such construct as may be desirable to provide embodiments of the present disclosure. Alternatively, the peptide and the bone-substitute scaffold are covalently bound. The term “osteoinduction”, where used herein refers to the act or process of stimulating osteogenesis. The term “osteogenesis”, as used herein refers the process of laying down new bone material by osteoblasts. The term “osteogenic” refers to agents or features which promote osteogenesis, directly or indirectly.

In certain embodiments, the present disclosure includes expression vectors capable of expressing one or more peptide molecules described herein. Expression vectors for different cell types are well known in the art and can be selected without undue experimentation. Generally, the DNA encoding the fusion polypeptide is inserted into an expression vector, such as a plasmid, in proper orientation and correct reading frame for expression. If necessary, the DNA may be linked to the appropriate transcriptional and translational regulatory control nucleotide sequences recognized by the desired host, although such controls are generally available in the expression vector. The vector is then introduced into the host through standard techniques. Guidance can be found e.g., in Sambrook et al. Molecular Cloning: A Laboratory Manual, 3d ed., Cold Spring Harbor Laboratory Press, NY 2001)).

In at least certain embodiments, the peptide conjugates of the present disclosure, whether wholly or partially synthetically or recombinantly produced, may be used as a pharmaceutical composition when combined with a pharmaceutically acceptable carrier. Such a composition may contain, in addition to the peptide conjugate and carrier, diluents, fillers, salts, buffers, stabilizers, solubilizers, and other materials well known in the art. Suitable carriers, vehicles and other components of the formulation are described, for example, in Remington: The Science and Practice of Pharmacy, 22^{nd} ed. The term “pharmaceutically acceptable” means a non-toxic material that does not interfere with the effectiveness of the biological activity of the peptide conjugate. The characteristics of the carrier will depend on the route of administration.

The pharmaceutical compositions of the present disclosure may be in the form of liposomes in which the peptide conjugate is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

In certain embodiments, an effective amount of the peptide described herein may be used for treatment of a particular condition which would benefit from the biological activity of the peptide. The effective amount can be determined by the attending diagnostician, as one skilled in the art, by the use of conventional techniques and by observing results obtained under analogous circumstances. In determining the therapeutically effective dose, a number of factors may be considered by the attending diagnostician, including, but not limited to: the species of the subject; its size, age, and general health; the response of the individual subject; the mode of administration; the bioavailability characteristic of the preparation administered; the dose regimen selected; the use of concomitant medication; and other relevant circumstances. An effective amount of a peptide of the present disclosure also refers to an amount of the peptide which is effective in controlling or reducing the particular condition.

An effective amount of a composition of the present disclosure will generally contain sufficient active ingredient (i.e., the peptide) to deliver from about 0.1 μg/kg to about 100 mg/kg (weight of active ingredient/body weight of patient). Particularly, the composition will deliver at least 0.5 μg/kg to 50 mg/kg, and more particularly at least 1 μg/kg to 10 mg/kg. Without wishing to be held to a specific dosage, it is contemplated that the various pharmaceutical compositions used to practice the method of the present disclosure may contain, but are not limited to, about 0.01 mg to about 25 mg of the peptide per kg body weight per dose.

Practice of the method of the present disclosure may include administering to a subject an effective amount of the peptide in any suitable systemic or local formulation, in an amount effective to deliver the dosages listed above, including but not limited to an implantable material such as a scaffold. In one embodiment, an effective, particular therapeutic dosage of the peptide is 1 μg/kg to 10 mg/kg. The dosage can be administered on a one-time basis, or (for example) from one to five times per day or once or twice per week, or continuously via a venous drip, depending on the desired therapeutic effect. In one therapeutic method of the present disclosure, the peptide is provided in an IV infusion in the range of from 1 mg/kg-10 mg/kg of body weight once a day. The duration of an intravenous therapy using the pharmaceutical composition of the present disclosure will vary, depending on the condition being treated and the condition and potential idiosyncratic response of each individual patient. In at least one embodiment, it is contemplated that the duration of each application of the peptide may be in the range of 1 to 4 hours and given once every 12 or 24 hours by continuous intravenous administration. Other therapeutic drugs, intravenous fluids, cardiovascular and respiratory support could also be provided if requested by the attending physician in a manner known to one of ordinary skill in the art.

In practicing the method of treatment or use of the peptides of the present disclosure, an effective amount of the peptide is administered to a mammal having a condition to be treated, such as a bone defect. The peptide may be administered in accordance with the method of the present disclosure either alone or in combination with other therapies.

Administration of the peptide used in the pharmaceutical composition or to practice the method of the present disclosure can be carried out in a variety of conventional ways, such as, but not limited to, orally, by inhalation, rectally, or by cutaneous, subcutaneous, intraperitoneal, vaginal, or intravenous injection, or via implantation of a scaffold or other implantable peptide-containing or peptide-coated structure.

When an effective amount of the peptide is administered orally, the compound may be in the form of a tablet, capsule, powder, solution or elixir. The pharmaceutical composition may additionally contain a solid carrier such as a gelatin or an adjuvant. The tablet, capsule, and powder particularly contains from about 0.05 to 95% of the peptide compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, 35 propylene glycol or polyethylene glycol. When administered in liquid form, the pharmaceutical composition particularly contains from about 0.005 to 95% by weight of peptide. For example, a dose of 10-1000 mg once to twice a day could be administered orally.

For oral administration, the peptide can be formulated into solid or liquid preparations such as capsules, pills, tablets, lozenges, melts, powders, suspensions, or emulsions. Solid unit dosage forms can be capsules of the ordinary gelatin type containing, for example, surfactants, lubricants and inert fillers such as lactose, sucrose, and cornstarch or they can be sustained release preparations.

In another embodiment, the peptide of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the peptide in an aqueous or non-aqueous pharmaceutically acceptable solvent which may also contain suspending agents, sweetening agents, flavoring agents, and preservative agents as are known in the art.

For parenteral administration, for example, the peptide may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives, and buffers as are known in the art.

When an effective amount of the peptide is administered by intravenous, cutaneous or subcutaneous injection, the peptide may be in the form of a pyrogen-free, parenterally acceptable aqueous solution or suspension. The preparation of such parenterally acceptable peptide solutions, having due regard to pH, isotonicity, stability, and the like, is within the skill in the art. A particular pharmaceutical composition for intravenous, cutaneous, or subcutaneous injection may contain, in addition to the peptide, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical compositions of the present disclosure may also contain stabilizers, preservatives, buffers, antioxidants, or other additive known to those of skill in the art.

As noted above, the compositions can also include an appropriate carrier. For surgical implantation, the peptide may be combined with any of the well-known biodegradable and bioerodible carriers, such as polylactic acid and collagen formulations. Such materials may be in the form of solid implants, sutures, sponges, wound dressings, and the like. In any event, for local use of the materials, the peptide is usually present in the carrier or excipient in a weight ratio of from about 1:1000 to 1:20,000, but is not limited to ratios within this range. Preparation of compositions for local use is detailed in Remington: The Science and Practice of Pharmacy, 22^{nd} ed.

As noted, particular amounts and modes of administration are able to be determined by one skilled in the art. One skilled in the art of preparing formulations can readily select the proper form and mode of administration depending upon the particular characteristics of the peptide conjugate selected, the condition to be treated, and other relevant circumstances using formulation technology known in the art, described, for example, in Remington: The Science and Practice of Pharmacy, 22^{nd} ed. The pharmaceutical compositions of the present disclosure can be manufactured utilizing techniques known in the art. As noted above, typically the effective amount of the peptide conjugate will be admixed with a pharmaceutically acceptable carrier.

Additional pharmaceutical methods may be employed to control the duration of action of the peptide. Increased half-life and controlled release preparations may be achieved through the use of polymers to conjugate, complex with, or absorb the peptide described herein. The controlled delivery and/or increased half-life may be achieved by selecting appropriate macromolecules (for example, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide, proteins (e.g., bovine serum albumin or human serum albumin) and the appropriate concentration of macromolecules as well as the methods of incorporation, in order to control release.

Another possible method useful in controlling the duration of action by controlled release preparations and half-life is incorporation of the peptide into particles of a polymeric material such as polyesters, polyamides, polyamino acids, hydrogels, poly(lactic acid), ethylene vinylacetate copolymers, copolymer micelles of, for example, PEG and poly(l-aspartamide).

It is also possible to entrap the peptide in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the peptide composition is to be used as an injectable material, it can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

In certain embodiments the peptide of the present disclosure can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

As mentioned above, the peptide of the present disclosure may be incorporated into pharmaceutical preparations which may be used for therapeutic purposes. However, the term “pharmaceutical preparation” is intended in a broader sense herein to include preparations containing a peptide composition in accordance with present disclosure, used not only for therapeutic purposes but also for reagent, imaging, or diagnostic purposes as known in the art. The pharmaceutical preparation intended for therapeutic use should contain a “pharmaceutically acceptable” or “effective amount” of the peptide compound, i.e., that amount necessary for a therapeutic response in a patient or subject in need of such treatment. If the pharmaceutical preparation is to be employed as a reagent, imaging, or diagnostic, then it should contain reagent, imaging, or diagnostic amounts of the peptide.

Peptides of the present disclosure include, but are not limited to, amino acid sequences which include the thirty-nine 12 mer amino acid sequences shown in Table 2, or variants of the peptides of Table 2 as discussed in further detail elsewhere herein. The peptide may optionally further comprise an amino acid linker sequence of, for example, 1 to 100 additional amino acids.

TABLE 2
Bone morphogenic protein receptor
binding peptides

| Peptide | | SEQ ID |
|---|---|---|
| No. | Amino Acid Sequence | NO: |
| 1 | TAKYLPMRPGPL | 1 |
| 2 | VHVPLHRGAVSA | 2 |
| 3 | SGTQDSMVGWNK | 3 |
| 4 | RDYHPRDHTATW | 4 |
| 5 | GNNPLHVHHDKR | 5 |
| 6 | TAKSLPMRPGPL | 6 |
| 7 | NAKYPTMRPGPK | 7 |
| 8 | DYHDPSLPTLRK | 8 |
| 9 | NAKYPTMRPGPL | 9 |
| 10 | TSKYLTMRPGPK | 10 |
| 11 | DYHDPSLPTVRK | 11 |
| 12 | GNHPPHVHHDQH | 12 |
| 13 | GNKSLHVHPGKR | 13 |
| 14 | NMKVATLHEYYN | 14 |
| 15 | NMKVATLNEYYN | 15 |
| 16 | GNNSLHVHPDQR | 16 |
| 17 | GAKSLPMRPGPL | 17 |
| 18 | GNKSLHVHHDKR | 18 |
| 19 | DYHYPSLPPVPK | 19 |
| 20 | NDKTGKAISRNQ | 20 |
| 21 | DYHDPSLLPVRK | 21 |
| 22 | DYHXPSLLPVRK | 22 |
| 23 | DWHDPSLLAVRK | 23 |
| 24 | GYKSPHLLPVPR | 24 |
| 25 | GKKSLPMRHDTR | 25 |
| 26 | SGTQDSMVGWNK | 26 |
| 27 | SPWLDMVELRRP | 27 |
| 28 | RHYQRRGEIFTG | 28 |
| 29 | HLEALSDLVNRN | 29 |
| 30 | ADRFCDMSQCAP | 30 |
| 31 | SFNWRYIEMSRW | 31 |
| 32 | SGIQDSMVGWNK | 32 |
| 33 | TAKYLPMRPGPL | 33 |
| 34 | RDYQPREHGGNR | 34 |
| 35 | RDFHPRXHXATW | 35 |
| 36 | DYHDPSLPTLRK | 36 |
| 37 | FDDVYWRWTYTA | 37 |
| 38 | QVNGLGERSQQM | 38 |
| 39 | SGLSSSWSYCYL | 39 |

Examples of drugs, reagents, imaging, and/or diagnostic agents that may be conjugated, directly or indirectly, to the peptides disclosed herein include, but are not limited to: (a) therapeutic drugs including those having suitable sites for linking directly to or via a linker to the peptide, wherein the drug has activity while still conjugated to the peptide, or becomes active upon release from the peptide at the binding site; for example the linker can be an acid-labile linker, an ester linker or a carbamate linker, (b) diagnostic reagents, such as radioisotopes for PET imaging, quantum dots, and near-infrared red excited dye for fluorescence imaging, and magnetic nanoparticles such as iron oxide for MRI, (c) oligonucleotides (DNA or RNA) or oligopeptides can be used as detectable or imaging reagent for diagnosis or for therapeutic use, (d) drug carriers including nano-carriers (nano-cages, nano-dendrites and etc.), liposomes, polymeric drug scaffolds and polymeric micelles those are able to load, transport and release drugs, and (e) functional particles such as gold-nanorods and gold-nanoparticles, or photosensitizer reagents such as aminolevulinic acid (ALA), and Silicon Phthalocyanine, for example, which are sensitive to either wide spectrum or specific wavelength laser that can be used for a desired therapy.

In at least one embodiment of the present disclosure, the peptide comprises an amino acid sequence which includes a 12-amino acid sequence selected from SEQ ID NO:1, 2, 3, 4, 5, 6, 7, 8, 9, or 10, or variants thereof. The peptide may optionally comprise an amino acid linker sequence of, for example, 1 to 100 additional amino acids such that the peptide comprises or consists of 13-112 total amino acids (e.g., 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids). In certain embodiments the active peptide (i.e., SEQ ID NO: 1, 2, 3, 4, 5, 6, 7, 8, 9, or 10, or variants thereof) plus the linker peptide comprises up to 50 amino acids, such that the linker peptide comprises 13, 14, 15, 16, 17, 18, 19, 20, 21, 22, 23, 24, 25, 26, 27, 28, 29, 30, 31, 32, 33, 34, 35, 36, 37, or 38 amino acids. The linker sequence may extend from the N-terminal or C-terminal end of the amino acid sequence SEQ ID NO: 1, 2, 3, 4, 5, 6, 7, 8, 9, or 10, or variants thereof. The amino acids of the linker sequence, or the substituted amino acids of the active peptide (SEQ ID NO: 1, 2, 3, 4, 5, 6, 7, 8, 9, or 10, or variants thereof) may be selected from, but are not limited to, the group consisting of gly, L-ala, L-arg, L-asn, L-asp, L-cys, L-glu, L-gln, L-his, L-ile, L-leu, L-lys, L-met, L-phe, L-pro, L-ser, L-thr, L-trp, L-tyr, L-val, D-ala, D-arg, D-asn, D-asp, D-cys, D-glu, D-gln, D-his, D-ile, D-leu, D-lys, D-met, D-phe, D-pro, D-ser, D-thr, D-trp, D-tyr, and D-val.

In certain embodiments, the peptides of the present disclosure include variants of SEQ ID NO: 1, 2, 3, 4, 5, 6, 7, 8, 9, or 10, which may include substitutions, such as conservative substitutions, or any amino acid in a D or L configuration (such as listed above), wherein the variant peptide has the activity of the non-variant version of the peptide. In alternate embodiments, the substituted amino acids may be selected from other non-natural amino acids including, but not limited to, those listed in U.S. Pat. No. 6,559,126, the entirety of which is hereby expressly incorporated by reference herein. Examples of substitutions include but are not limited to those described elsewhere herein. In at least certain embodiments, the variant peptides of SEQ ID NO: 1, 2, 3, 4, 5, 6, 7, 8, 9, or 10, comprises at least at least 91% sequence identity (as defined elsewhere herein) with SEQ ID NO: 1, 2, 3, 4, 5, 6, 7, 8, 9, or 10, i.e., it is identical except for one amino acid substitution. In at least certain embodiments, the variant peptides of SEQ ID NO: 1, 2, 3, 4, 5, 6, 7, 8, 9, or 10, comprises at least at least 83% or greater sequence identity (as defined elsewhere herein) with SEQ ID NO: 1, 2, 3, 4, 5, 6, 7, 8, 9, or 10, i.e., it is identical except for one or two amino acid substitutions. In at least certain embodiments, the variant peptides of SEQ ID NO:1, 2, 3, 4, 5, 6, 7, 8, 9, or 10, comprises at least 75% or greater sequence identity (as defined elsewhere herein) with SEQ ID NO:1 2, 3, 4, 5, 6, 7, 8, 9, or 10, respectively, i.e., it is identical except for, for example, one, two, or three amino acid substitutions.

The scaffolds and implants which are coated with or contain the peptides disclosed herein may be made from any of a variety of materials well known in the orthopedic implant arts. Examples of materials that can be used to construct the scaffolds used in the methods of the present disclosure include metals and titanium alloys such as, but not limited to, those having the following approximate compositions, Ti-6Al-4V, Ti-7.5Mo, Ti-15Mo, Ti-6Al-7Nb, Ti-10Nb-10Zr, Ti-4Ta-4Sn, Ti-13 Nb-13Zr, Ti-12Mo-6Zr-2Fe, Ti-5Al-2.5 Fe, Ti-15Mo-5Zr-3Al, Ti-15Zr-4Nb-2Ta-0.2Pd, Ti-29Nb-13Ta-4.6Zr, Nitinol (Ni—Ti alloy), Titanium, Tantalum, and stainless steel. Other examples of materials that can be used to construct the scaffolds used in the methods of the present disclosure include, but are not limited to, natural cancellous bone, demineralized natural cancellous bone, natural polymers (fibrin and collagen), synthetic polymers (polycarbonates, polyanhidrides, poly(ethylene oxide), polyfumarates, poly(lactic acid) (PLA), poly(glycolic acid) (PGA), and poly(lactic-co-glycolic acid) (PLGA), PEEK (polyetheretherketone), and polyphosphazene), calcium phosphate, hydroxyapatite, ceramics, and hydrogels (e.g., collagen hydrogel). In general, The scaffold materials used for implants herein may comprise any material suitable material known in the art for making such implants including such materials as ceramics, metals, and polymers, including materials that slowly degrade as the scaffold material is converted over time into bone.

Implants of the present disclosure may be made from the various materials such as by being machined therefrom, but alternatively, may be manufactured by injection molding or three-dimensional (3-D) lithographic printing, for example. When manufactured by three-dimensional lithographic printing, the scaffold may be made of polymers, such as PEEK or other polymer and/or absorbable materials such as tri-calcium phosphate (TCP), hydroxyapatite (HA) or the like. When made of metal, the scaffold may be machined or made by metal powder deposition, for example. Alternatively, as noted, the scaffold may be made of PEKK (poly(oxy-p-phenyleneisophthaloyl-phenylene/oxy-p-phenylenetere-phthaloyl-p-phenylene) or carbon-filled PEEK.

In various embodiments, the peptide and the implant (e.g., scaffold or semi-solid material such as a hydrogel) are non-covalently bound, for example ionically. For example, peptide-bearing phage viruses may be linked via negative charge to an implant having a surface coated with positively-charged polylysine linker molecules (or other positively-charged linker molecule). Alternatively, the peptides could be linked directly to a linker molecule on the implant surface, e.g., peptides with a negative charge could be ionically-linked to positively-charged polylysine linker molecules (or other positively-charged linker molecule) on the implant surface. Alternatively, the peptides may be linked to the implant via covalent bonds.

Other strategies for attaching the peptides to the scaffold include, but are not limited to, linking the osteogenic peptides to a binding peptide extending from the scaffold. This allows one end of the fused peptide to bind to the scaffold (e.g. to titanium) and the other end free to enable the triggering of bone regeneration. In another embodiment, a polydopamine (PDOP) coating can be applied to the scaffold (e.g., 2 mg/mL dopamine hydrochloride, 10 mM Tris-buffer, pH 8.5, dark, 12 h, room temperature) to immobilize BMPR-binding peptides via electrostatic surface adsorption. If surface adsorption is not stable, 1,2-Dioleoyl-sn-glycero-3-phosphocholine (DOPC)-modified scaffolds may be converted to possess azide groups (using bromoisobutyryl bromide and sodium azide) for click coupling to propargyl-modified (done by conjugating proparylamine to the free C-terminus) BMPR-binding peptides. Alternatively, BMPR-binding peptides may be anchored to 3-aminopropyltriethoxysilane (APTES)-modified Ti-scaffolds through a hetero-bifunctional H₂N-PEG-CO₂H linker via EDC/NHS chemistry.

In various embodiments, the implant may be seeded with cells, such as MSCs (e.g., hMSCs when the subject to be treated is a human).

As discussed hereinabove and in the examples, peptides as disclosed herein are useful as stimulators of growth and development of cells and tissues involved in bone formation. Without being limited by any particular theory or mechanism of action, compounds herein such as peptides are capable of function on a large range of cell types, for example, to stimulate the healing of bone defects, for example, defects in weight-bearing bones such as fractures, and bones of the jaw having defects associated with progressive gum diseases and tooth loss. Peptides disclosed herein may further be used to stimulate bone re-growth under physiological conditions of bone loss such as osteopenia and osteoporosis. These conditions are increasingly found in aging populations particularly in temperate climates in which vitamin D seasonally limits extent of bone formation and bone metabolism is consequently shifted to osteoclastic bone destruction. Alternatively, peptides herein are effective in increasing incorporation of bone grafts following surgery to reconstruct bones after traumatic events such as collisions and war injuries. Suitable bone grafts include, but are not limited to autografts, artificial bone grafts, allografts, delayed grafts, full thickness grafts, heterologous grafts, xenografts, homologous grafts, and hyperplastic grafts.

The three-dimensional lattice configuration of certain scaffold embodiments of the present invention may be adapted for use in a variety of orthopedic applications of the skeleton. For example, intramedullary nails for use in long bones such as the femur, tibia, fibula, radius or ulna may be constructed using the peptide-coated scaffolds of the present disclosure, to provide an anisotropic characteristic such as modulus, to match that of the native surrounding environment. Extra medullary implants, such as plates, screws, spacers, rods, sacroiliac joint fusion implants or others may also be constructed utilizing the 3-D printed structures described herein.

The implants disclosed herein may be provided with a porous or textured surface, such as to facilitate osteogenesis or in the case of porous surfaces, to elute drugs such as antibiotics, anticoagulants, bone growth factors or others known in the art. Implants produced in accordance with the present disclosure may comprise hybrid constructs, with a first component made from 3-D printed structure and a second component molded, machined or otherwise formed from a conventional implant material such as titanium, various metal alloys, PEEK, PEBAX, or others well known in the art, and/or described elsewhere herein.

Various embodiments of the present disclosure will be more readily understood by reference to the following examples and description, which as noted above are included merely for purposes of illustration of certain aspects and embodiments of the present disclosure, and are not intended to be limiting. The following detailed examples and methods describe how to make and use various peptides, peptide conjugates, peptide compositions, and peptide-coated scaffolds of the present disclosure and are to be construed, as noted above, only as illustrative, and not limitations of the disclosure in any way whatsoever. Those skilled in the art will promptly recognize appropriate variations from the materials and procedures described herein.

### EXAMPLES

Methods and results of non-limiting examples and embodiments of the present disclosure are summarized below. In particular, as explained in more detail below, the 12-mer peptides TAKYLPMRPGPL (SEQ ID NO:1), VHVPLHRGAVSA (SEQ ID NO:2), SGTQDSMVGWNK (SEQ ID NO:3), and RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10) among others, were discovered to bind the bone morphogenic protein receptors BMPR1A and BMPR2. These peptides, genetically displayed on the pVIII protein of M13 phage, were assembled into a phage film. Human mesenchymal stem cells, when cultured on this film, differentiated into to osteoblast cells. Additionally, 3D printed scaffolds (such as made of titanium alloy) that were coated with one or more of the peptides (e.g., by a process called double sided freeze casting) demonstrated bone regeneration in vivo.

### Example 1

Biopanning for Discovery of Peptides Capable of Binding and Detecting Bone Morphogenic Protein Receptors

M13 phage is a human-safe natural biopolymer nanofiber composed of ˜3000 highly ordered copies of the major coat protein (p8, made of 50 amino acids) surrounding a circular ssDNA. The DNA encodes coat proteins including p8 on the major coat (i.e., side wall) and four other structural proteins at the two tips (called minor coats) such as p3. By inserting DNA encoding peptides into the genes of the coat proteins, the peptides are themselves displayed on the outer surface of the phage at the tips by genetic fusion to minor coat proteins (e.g., p3 display) or along the length by genetic fusion to major coat protein p8 (p8 display). Thus, a library made of billions of phage clones with each clone displaying a unique foreign peptide sequence can be constructed. Currently, there are two major types of peptide libraries: (1) a p3-displayed phage library where each peptide is displayed at the p3 tip through fusion to 5 copies of p3, and (2) a p8-displayed phage library where each peptide is displayed along the side wall through fusion to every copy of p8. The p3-displayed phage library has been used to identify a peptide sequence that can specifically recognize and bind to a target (e.g., a protein, a cell, or a crystal) through biopanning Herein, a p3-displayed phage library provided a pool of peptide candidates to screen the peptides that can target and bind the extracellular domain of the bone morphogenic protein (BMP) receptors (BMPR1A and BMPR2).

Currently there is a need to identify short peptide sequences derived from BMP-2 that can induce or promote the osteogenic differentiation. Phage display can identify BMPR1A and BMPR2-binding peptide domains. Namely, BMPs bind and recognize BMP receptors (BMPR1A and BMPR2) to trigger the osteogenic differentiation. Hence, BMP receptor-binding peptides may potentially mimic BMPs in inducing osteogenic differentiation. Short peptides possess several advantages in biomedical applications compared to proteins. Peptides can be easily synthesized in high yields, high purities, and at low costs. Compared to proteins, small molecular weight peptides (2000 Da) can enter cells and penetrate tissue efficiently and have a lower immunogenicity. Peptides also do not need to be produced in animals, overcoming burdensome manufacturing conditions as well as ethical issues.

Materials

Various chemicals and materials used herein included agarose, Anti-M13 Bacteriophage Coat Protein g8p antibody [RL-ph2] (ab9225), azino-bis(3-ethylbenzothiazole sulfonic acid) diammonium salt (Sigma, cat. #A-1888), BMPR1A-Fc (G&P Biosciences), BMPR2-Fc (G&P Biosciences), Bovine Serum Albumin, 5-Bromo-4-chloro-3-indolyl-β-D-galactoside (Xgal), Corning® BioCoat™ Poly-D-Lysine Multiwell and Assay Plates (Thomas Scientific), dimethyl formamide, E. coli host strain ER2738 (New England Biolabs), Glycine-HCl, Goat-anti mouse AF-488 (Abcam), isopropyl-β-D-thiogalactoside (IPTG), Ethanol (Sigma-Aldrich), Human Mesenchymal Stem Cells (Lonza), Luria Broth Medium, Mesenchymal stem cell growth mediaBulletKit (PT-3238 & PT-4105) (Lonza), mouse anti-g3p (p8) IgG, Ph.D.™-12 Phage Display Peptide Library Kit (New England Biolabs), phosphate buffered saline, polyethylene glycol-8000 (PEG), PureBlu™ Hoechst 33342 Nuclear Staining Dye #1351304 (BioRad), Sodium Azide, Sodium Bicarbonate, Sodium Chloride, Tetracycline, 3,3′,5,5′-Tetramethylbenzidine, and Tris-HCl.

Negative Selection of Plastic Binders

The Ph.D.™-12 phage library was diluted in tris-buffered saline mixed with Tween-20 (TBS+0.1% v/v Tween-20 and abbreviated TBST) for a final concentration of 2×10¹¹ phage in 1 mL of TBST. The diluted library was then pipetted onto one of the wells of a 12-well poly-L-lysine plate followed by rocking the plate gently for 60 minutes at room temperature. Next, the supernatant containing phages which did not bind to either the plate or poly-L-lysine were collected. This supernatant served as the starting phage library for subsequent binding selections. The phages which stuck to the plate were discarded.

Immobilization of Target

This procedure is only applicable if the charge of the desired protein to be immobilized is negative, determined by checking the pI value of the protein. When the pH a protein has a net negative charge. This procedure was written for 12-well plates coated in poly-L-lysine (positively charged). Separate solutions of BMPR1A-Fc and BMPR2-Fc were prepared at 80 μg/ml in Tris-buffered saline (pH 7.5). In separate wells and on separate poly-L-lysine coated 12-well plates, 0.3 ml of each solution of receptor was added. The solutions of BMPR1A-Fc and BMPR2-Fc were swirled around the plate surface until the entire surface was wet. The plates were then incubated overnight at 4° C. with gentle agitation in a humidified container (a sealed plastic box with damp paper towels). Each individual receptor was now immobilized.

Selection Procedure

The following selection procedure is based off of the direct coating procedure outlined in the Ph.D.™-12 Phage Display Libraries instructional manual (New England Biolabs) with some alterations. This procedure was carried out after immobilizing the targets BMPR1A-Fc and BMPR2-Fc in a 12-well poly-L-lysine coated plate. A stock solution of tetracycline was prepared (20 mg/ml in 1:1 ethanol:water). The coating solution containing the target receptors was poured off followed by firmly slapping the plates face down on a clean paper towel. Each well was then filled with blocking buffer (0.1 M NaHCO₃ (pH 8.6), 5 mg/mL BSA, 0.02% NaN₃. Filter sterilize, store at 4° C.) and incubated for 1 hour at 4° C. The blocking solution was then poured off followed by firmly slapping the plates down against a clean paper towel. Each plate was washed rapidly 6 times with Tris-buffered saline mixed with Tween-20 (TBS+0.1% v/v Tween-20). The plate was slapped against a clean paper towel each time. 1 ml of the phage library created from the negative selection process was then pipetted onto each of the target coated wells. The plates were then rocked gently at room temperature for 60 min. The non-binding phages were then discarded by pouring off the supernatant and slapping the plate face-down on a clean paper towel. The plate was then washed 10 times with TBST followed by slapping the plate face-down on a clean paper towel each time (a clean section of paper towel was used each time to prevent cross-contamination). The bound phages were then eluted by incubating with a nonspecific disruption buffer (0.2 M Glycine-HCl (pH 2.2), 1 mg/ml bovine serum albumin (BSA) for 15 minutes. The eluate (immersed in the disruption buffer) was then pipetted into a microcentrifuge tube followed by neutralization with 150 μl of 1 M Tris-HCl (pH 9.1). A small portion (˜1 μl) of this eluate could be titered and sequenced if desired. The rest of the eluate was amplified (see amplification of phage library section). Once amplified, the eluate was also titered. Determine the concentration of amplified phages by counting the blue plaques in titering as described. Dilute the amplified library to contain 2×10¹¹ phages in 1 mL of TBS (as little as 10⁹ plaque forming units per ml can be used if there is not enough). A second poly-L-lysine 12-well plate was then coated with the target receptors to allow for a second round of biopanning carried out using the same steps except that the Tween-20 concentration in the wash steps was raised to 0.5% (v/v). The eluate from the second round was then amplified, titered and used in an additional 3rd round of panning on newly receptor coated plates. 0.5% Tween was again used in the washing steps for the third round, after which the eluate was titered. Blue plaques from the titering were picked and incubated in 10 ml of luria broth overnight. The cultures were then mixed with an equal amount of culture: 50% glycerol in water and stored at −80° C. or sent on ice for sequencing.

Titering

The E. coli strain ER2738 was inoculated into 10 ml of luria broth and tetracycline (final concentration of 12 μg/ml tetracycline). The culture was incubated at 37° C. with vigorous shaking. This culture was grown to an early log phase of growth (optical density at 600 nm was between 0.01-0.05). The top agar was melted in the microwave and 3 ml was transferred to a sterile culture tube. One tube was prepared for each phage dilution. The phage dilutions of the amplified panning eluate were typically from 10⁸-10¹⁰ pfu and they were 10¹-10⁴ pfu for unamplified eluate. The early log phase E. coli culture was then dispensed into separate 200 μl microcentrifuge tubes (one for each phage dilution). The cultures were then inoculated with 10 μl of phage solution for each individual dilution. The inoculated culture was allowed to incubate at room temperature for 1-5 min. The infected cells were then transferred one infection at a time to the culture tubes containing 45° C. Top Agar. The tubes were vortexed briefly and immediately poured onto prewarmed LB/IPTG/Xgal plates prepared according to the Ph.D.™-12 Phage Display Libraries instructional manual (New England Biolabs). The plates were gently tilted to spread the top agar out evenly. The plates were then allowed to cool for 5 min at room temperature, inverted, and incubated overnight at 37° C. On the next day, the blue plaques could be counted or picked for sequencing. For plates that had roughly 100 plaques, the plaques were counted and the number of plaques was multiplied by the dilution factor to get the plaque forming units per 10 μl.

Amplification of Phage Library

The E. coli strain ER2738 was inoculated into 20 ml of luria broth medium in a 250 ml Erlenmeyer flask. The culture was incubated at 37° C. with vigorous shaking. The eluate not used for titering from the previous round of selection (depending on which round was last completed) was added to the 20 ml ER2738 culture during the early-log phase of growth (optical density at 600 nm was between 0.01-0.05). The culture was then incubated for 4.5 hours at 37° C. with vigorous shaking. The culture was then transferred to a centrifuge tube and spun for 10 min at 12,000 g at a temperature of 4° C. The supernatant was then transferred to a new tube and re-spun. The upper 80% of the supernatant was then transferred to a new tube and mixed with 1/6 volume 20% PEG/2.5 M NaCl. The phages were then allowed to precipitate out at 4° C. overnight (or a minimum of 2 hours). The PEG precipitated phages were then centrifuged at 12,000 g for 15 min at 4° C. The supernatant was discarded and the tube was re-spun briefly. Any residual supernatant was removed with a pipette. The phage pellet was then visible as a very small white finger print like smear on the side of the tube. The pellet was then resuspended in 1 mL of TBS. The resuspended phages were then centrifuged at 14,000 rpm for 5 min at 4° C. to remove any residual cells. The supernatant was then transferred to a new microcentrifuge tube followed by reprecipitation in 1/6 volume 20% PEG/2.5 M NaCl. The solution was incubated on ice for 60 min. The solution was then microcentrifuged on a table top centrifuge at 14,000 rpm for 10 min at 4° C. The supernatant was again discarded, and the tube was spun briefly followed by removal of any additional supernatant. The pellet was then resuspended in 200 μL of TBS. The phage solution was then re-spun for 1 mi to remove any insoluble material. The supernatant was transferred to a fresh tube. This solution was considered the amplified eluate.

Peptide Sequencing

Blue plaques from the titering were picked and incubated in 10 ml of luria broth overnight. The cultures were then mixed with an equal amount of culture: 50% glycerol in water and stored at −80° C. or sent on ice for sequencing. Those samples sent for sequencing were sent to MCLAB Molecular Cloning Laboratories. The pIII coat protein gene for M13 phage was sequenced. The resulting chromatogram files containing the sequencing data were analyzed using FinchTV software. The peptide sequences could be determined according to the Ph.D.™-12 Phage Display Libraries instructional manual (New England Biolabs).

Enzyme-Linked Immune Sorbent Assay for Receptor Binding

Blocking buffer (0.1 M NaHCO₃ (pH 8.6), 5 mg/ml BSA. Filter sterilized, stored at 4° C.) was prepared. TBS (50 mM Tris-HCl (pH 7.5), 150 mM NaCl, autoclaved, stored at room temperature) was prepared. Separate solutions of the target receptors were then made. For BMPR1A, a 10 μg/ml stock solution was made. For BMPR2, a 10 μg/ml solution was also made. Separate wells with n=4 for each different peptide bearing phage group were then coated with one of the receptors. The coating was done by adding 100 μl of the target solution to wells of a 96-well plate followed by swirling until the surface was completely wet. The plates were then allowed to incubate overnight at 4° C. with gentle agitation in a humidified container (a plastic box lined with damp paper towels). Any excess target solution was then removed, and the plate was slapped face-down on a clean paper towel. The wells of the plate were then blocked with 200 μl of blocking buffer for 1 hour at room temperature. An additional row of uncoated wells was also blocked to serve as a background signal control. The blocking buffer was then removed from each plate followed by 5 washes with 200 μl of TBST. Each time the plate was slapped against a clean section of paper towel face-down. In a separately prepared blocked plate, serial dilutions of the phages in 200 μl of TBST were done. After a brief pilot test, it should be noted that using 10¹⁰ phage/ml was determined to work best, but other concentrations of virus also worked consistently. Therefore, 100 μL of 10¹⁰ phage/ml was pipetted (with a multichannel pipettor) to each row of target-coated wells as well as the rows without target. The mixtures were incubated at room temperature for 1.5 hours with agitation. The wells were then emptied and washed 5 times with 200 μl of TB ST. Next, 100 μl of mouse anti-g3p (p8) IgG was added to every well followed by incubation at room temperature for 1 hour. The wells were then washed 5 times with 200 μl of TB ST. Then, 100 pI of anti-mouse IgG conjugated with HRP (diluted in TBST+5% BSA) was added to every well followed by incubation for 1 hour. The wells were emptied followed by 5 more washes with 200 pI of TBST. Then, 100 pI of freshly prepared 3,3′,5,5′-Tetramethylbenzidine (TMB) was added to each well and the absorbance at 370 nm and 652 nm was monitored for 1 hour using an ELISA plate reader. After 1 hour, the substrate reaction was stopped using equal volumes (100 pI) of 1 M HCl. The absorbance at 450 nm for each well was then measured.

Live Cell Binding Through Immunofluorescence

Human mesenchymal stem cells (hMSCs) were grown to roughly 60% plate coverage in a 48-well tissue culture plate according to the company Lonza's culturing protocols. Phages (each type having a different peptide displayed on the pIII coat protein from biopanning) were then filtered (0.2 μm syringe filter) and subjected to 1 hour of ultraviolet (UV) light for disinfection. The hMSCs were then blocked with 0.5% BSA (dissolved in cell media) for 1 hour. The cell media for the hMSCs was then replaced with 1 ml of new cell media having of concentration of 10¹² phages/ml. Next, the plates were washed once with 400 μl of phosphate buffered saline (PBS) at a pH of 7.4. The hMSCs were then incubated with Anti-M13 Bacteriophage Coat Protein g8p antibody [RL-ph2] (ab9225) at a 1:1000 dilution in cell media (200 μl per well). The wells were then washed again with PBS (400 μl per wash). Next, the hMSCs were incubated with the secondary antibody, Goat-anti mouse AF-488 (Abcam) for 1 hour (1 ug/ml final concentration of antibody in media). The wells were then washed again with PBS (400 μl per wash). A Hoechst nuclear stain was used to stain the nuclei (PureBlu™ Hoechst 33342 Nuclear Staining Dye #1351304 from BioRad) according to the company's protocol. Cell Mask™ Orange Plasma Membrane Stain was used to stain membranes (CellMask™ Orange Plasma Membrane Stain Catalog number: C10045 from Thermo Fisher Scientific) according to the company's protocol. The phages binding to the cells were then imaged on a Nikon Eclipse Ti fluorescent microscope.

Results

Sequencing Results and Frequency of Peptides

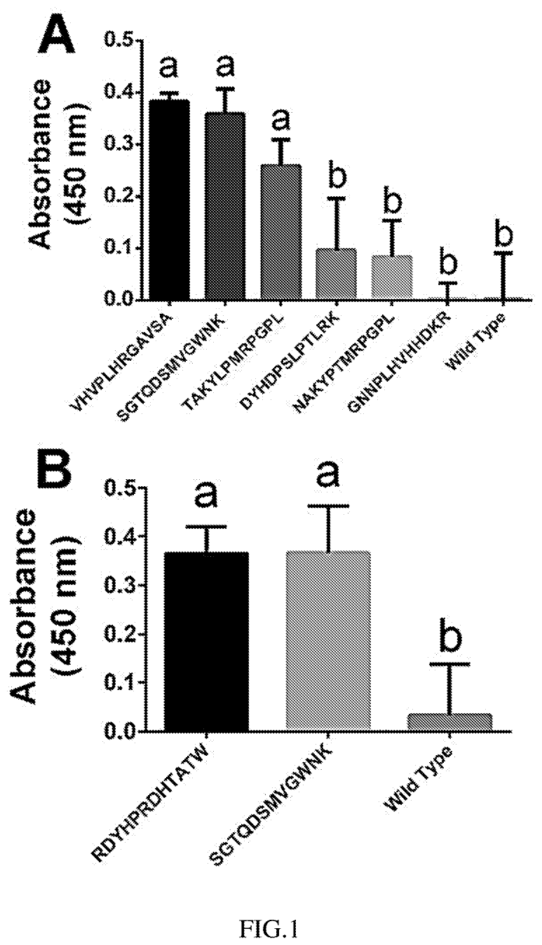

Upon sequencing the phage library used to biopan on the receptors BMPR1A and BMPR2, the DNA sequences were converted into peptide sequences for the peptide insert region of the DNA. The resulting frequencies and sequences of approximately 40 peptides are shown in FIG. 1 of Provisional Application Ser. No. 62/655,340. Each tick mark on the frequency axis represents a plaque picked from biopanning which was sequenced. For the receptor BMPR1A, the most frequently obtained peptide was TAKYLPMRPGPL (SEQ ID NO:1). For the receptor BMPR2, RDYHPRDHTATW (SEQ ID NO:4) was the most frequent peptide. While a higher frequency may indicate better binding to the receptors, it can also represent a preference in the ability of a phage to replicate during the amplification steps. Therefore, frequency alone is not a reliable indicator of the best peptides to select. Knowing this, both the highest frequency peptides and peptides which showed more unique sequences were selected for further tests. DNA sequences for the four peptides selected for further work were ACTGCGAAGTATCTGCCTATGCGTCCTGGGCCGCTT (SEQ ID NO:40), which encodes SEQ ID NO:1, GTTCATGTTCCGTTGCATAGGGGTGCGGTTTCGGCG (SEQ ID NO:41), which encodes SEQ ID NO:2, TCTGGTACTTAGGATTCTATGGTTGGTTGGAATAAG (SEQ ID NO:42), which encodes SEQ ID NO: 3, and CGGGATTATCATCCTCGTGATCATACTGCTACTTGG (SEQ ID NO:43), which encodes SEQ ID NO:4.

Relative Binding Strengths of Selected Peptides and Live Cell Binding of Peptides to hMSCs

Relative binding strength in an ELISA of several peptides are shown in FIG. 1. For the receptor BMPR1A, 3 statistically significant peptides emerged as the best binders. These peptides were VHVPLHRGAVSA, SGTQDSMVGWNK, and TAKYLPMRPGPL. For the receptor BMPR2, the sequences RDYHPRDHTATW and SGTQDSMVGWNK were statistically significant binders. The peptide SGTQDSMVGWNK was especially interesting because it showed up as a result of biopanning on both BMPR1A alone and BMPR2 alone. Additionally, live hMSC interactions with peptide-bearing phages were observed through immunofluorescence. The phages for the sequences SGTQDSMVGWNK, RDYHPRDHTATW, and VHVPLHRGAVSA bound to what appeared to be the same location as the outer membrane. The receptors are transmembrane receptors, and therefore it was expected that the binding would occur on the outer membrane as was observed.

In summary, biopanning was used to isolate M13 phages bearing peptides capable of binding to the receptors BMPR1A and BMPR2. These peptide-bearing phages were demonstrated to bind significantly better than a wild type phage control in ELISA tests. The peptides SGTQDSMVGWNK, RDYHPRDHTATW, and VHVPLHRGAVSA also demonstrated an ability to bind to the outer membrane (presumably on the BMPR1A and BMPR2 receptors) of live hMSCs.

### Example 2

Evaluation of BMPR-Binding Peptides

Genetic Manipulation of M13 Phages

Filamentous temperate M13 phages can be pictured as long flexible nanofibers composed of five structural capsid proteins, which encase a relatively large circular single strand of DNA. Wild-type M13 phage measures ˜9300 Å long, but has a diameter of only 65 Å. The M13 virion consists of ˜6407 bases of circular single-stranded DNA (ssDNA) in its genome, but longer genomes generate longer phages. Although there are five different proteins, the majority of a filamentous phage is coated by ˜2700 copies of a protein encoded by a single gene called pVIII, which is helically arranged to form a filament tube. This is the protein that is often modified for desirable characteristics. The ends of filamentous phages are composed of minor coat proteins. One end is composed of five copies each of the proteins pIII and pVI, while the opposite end displays five copies each of the proteins pVII and pIX. The filamentous structure and major coat proteins are what makes these phages capable of creating many self-assembled nanostructures as well as develop excellent targeting abilities. By inserting DNA encoding peptides into the genes of the coat proteins, the peptides are themselves displayed on the outer surface of the phages at the tips by genetic fusion to minor coat proteins (e.g., pIII display) or along the length by genetic fusion to major coat protein pVIII (pVIII display).

M13 bacteriophages have been implemented in a variety of organic synthesis strategies and organic biomaterials. One of the major biomaterials made out of M13 phages is called phage film. Phage films consist of self-assembled layers of M13 phages that can come in a variety of nanotopographies depending on the preparation method. Often times, the phages are naturally self-assembled into a ridge groove structure with the help of a pulling force. However, due to the liquid crystalline properties of M13 bacteriophage, even more complex structures can be made. A major benefit of phage films is that different peptides can be displayed on the phages without changing the structure of the film. This enables the effects of different peptides to be distinguished in cellular differentiation assays.

Osteoblasts, the cells responsible for the synthesis and secretion of the majority of the proteins in the bone extracellular matrix, are essential for new bone formation. Osteoblasts also induce mineralization of the extracellular matrix. Osteoblasts are derived from undifferentiated mesenchymal stem cells. These mesenchymal stem cells have the capacity to differentiate into osteoblasts, adipocytes, chondrocytes, and myoblasts. There are several frequently used markers for identifying osteoblasts. Some common markers include alkaline phosphatase, type I collagen, osteopontin, bone sialoprotein, and osteocalcin. However, the expression of these markers can vary over time. For example, alkaline phosphatase, bone sialoprotein, and collagen type 1a can be early indicators of osteoblast differentiation while osteocalcin is a late marker. Osteopontin expression peaks twice during proliferation and can later peak again during the late stages of differentiation. None of the genes mentioned which are involved in the extra cellular matrix mineralization are unique to osteoblasts or bone. However, the coexpression of these genes is unique to osteoblasts. Therefore, several of these markers and calcium mineralization were used in the following work to establish osteoblast differentiation.

Materials

Various chemicals and materials used herein included: Acetic acid, agarose, AlamarBlue® (Bio-Rad), Calcium Chloride, Chloramphenicol, deoxynucleosidetriphosphate, E. coli strain TG1, ER2738 bacteria (New England Biolabs), Ethylenediaminetetraacetic acid (EDTA), 5×GC buffer, Glycerol, Goat Anti-Rabbit IgG H&L Alexa Fluor 555ab150078 (Abcam), Goat Anti-Mouse IgG H&L (Dylight 650) preadsorbed ab96882 (Abcam), human Mesenchymal stem cell (hMSC) Osteogenic BulletKit (Lonza), Hind III restriction enzyme (New England Biolabs), human collagen type 1A primer (GeneCopoeia), human GAPDH primer (GeneCopoeia), human osteocalcin primer (GeneCopoeia), human osteopontin primer (GeneCopoeia), Luria broth, M13KO7 phage (New England Biolabs), Mesenchymal stem cell growth medium BulletKit (Lonza), MiniOpticon Real Time PCR System (Bio-Rad), Nco I restriction enzyme (New England Biolabs), OCN [OC4-30] ab13418 (Abcam), Pfu 1U DNA polymerase, polyethylene glycol-8000, Power SYBR™ Green Cells-to-CT™ Kit (Thermo Fisher Scientific), QIAgen gel DNA extraction kit, QIAgen miniprep plasmid extraction kit, RbpAB to osteopontin ab8448 (Abcam), T4 ligase (Invitrogen), Tris base, XL_1 Blue E. coli harboring the PeCan49 phagemid DNA for chloramphenicol resistance, and uranyl acetate.

Preparation of M13 Plasmid Vector

A culture of XL_1 Blue E. Coli harboring the PeCan49 phagemid DNA for chloramphenicol resistance was incubated at 37° C. overnight on a luria broth plate (35 μg/mi chloramphenicol antibiotics). The well separated clone from the overnight culture was then inoculated into 3 ml of luria broth medium with the antibiotic chloramphenicol and incubated overnight at 250 rpm at 37° C. Phagemid DNA was then isolated and prepared by a QIAgen miniprep plasmid extraction kit, and the DNA was eluted with 50 pI of sterile double distilled water (ddH₂O).

Linear vector fragments were prepared by digesting the phagemid DNA with both the Nco I and Hind III restriction enzymes purchased from New England Biolabs. To accomplish this, a sterile micro-centrifuge tube had the following components: Phagemid DNA (5 pI), 10× New England Biolab buffer 2 (2 pI), Nco I 10 U (1 pI), Hind III 10 U (1 pI), and ddH₂O 11 pI for a total of 20 pI of solution. The contents of the micro-centrifuge tube were then incubated for 2.5 hours at 37° C. After the digestion, the linear fragments were loaded into 1% agarose gel and isolated by electrophoresis in 1×TAE buffer (a mixture of Tris base, acetic acid and EDTA). The linear DNA fragments were extracted from the agarose gel using a QIAgen gel DNA extraction kit. The DNA was then eluted with 20 pI of sterile ddH₂O and kept at −20° C.

PCR Reaction (Preparation of Insert Fragments)

The polymerase chain reaction (PCR) was carried out in a sterile 200 pI micro-centrifuge tube using a Bio-RAD MJ Mini™ Personal Thermal Cycler. The reaction system consisted of M13KO7 phage RF DNA (2 μL) as the DNA template, primer 1 (1 μL at 50 pmol), primer 2 (1 μL at 50 pmol), 10 mM deoxynucleoside triphosphate (dNTP)(1 μL), 5×GC buffer (10 μL), ddH2O (35 μL), and Pfu 1U DNA polymerase (0.5 μL) under the PCR conditions of 98° C. for 30 sec, 30 cycles (98° C. for 10 sec, 56° C. for 30 sec and 72° C. for 15 sec), and a last extension at 72° C. for 4 minutes. Forward primers (primer 1) used were: 5′ ATCCATGGCGGTTCATGTTCCGTTGCATAGGGGTGCGGTTTCGGCGGATCCCGC AAAAGCG3′ (SEQ ID NO:44) for VHVPLHRGAVSA; 5′ ATCCATGGCGTCTGGTACTTAGGATTCTATGGTTGGTTGGAATAAGGATCCCGC AAAAGCG3′ (SEQ ID NO:45) for SGTQDSMVGWNK; 5′ ATCCATGGCGCGGGATTATCATCCTCGTGATCATACTGCTACTTGGGATCCCGC AAAAGCG-3′ (SEQ ID NO:46) for RDYHPRDHTATW; 5′ ATCCATGGCGACTGCGAAGTATCTGCCTATGCGTCCTGGGCCGCTTGATCCCGC AAAAGCG3′ (SEQ ID NO:47) for TAKYLPMRPGPL. The reverse primer (primer 2) used for all sequences was 5′ GCAAGCTTTTATCAGCTTGCTTTCGAG3′ (SEQ ID NO:48).

PCR Products Purification and Digestion

PCR products were purified using a QIAgen PCR product purification kit. The DNA was eluted with 10 pI of sterile ddH₂O. The purified DNA was then digested with both the Nco I and Hind III restriction enzymes in a sterile microcentrifuge tube. Next, the microcentrifuge tube was incubated for 2.5 hours at 37° C. After this digestion, the DNA fragments were loaded into 1% agarose gel and isolated by electrophoresis in 1×TAE buffer. The digested fragment of DNA was removed from the agarose gel by gel extraction. The DNA was then eluted with 20 pI of sterile ddH₂O.

Preparation of TG1 Competent Cells

An overnight culture of E. coli strain TG1 was grown in a 125 ml sterile flask at 37° C. with vigorous shaking to an optical density of −0.4 at 600 nm. The cells were then collected by centrifuging at 2500 rpm at a temperature of 4° C. for 10 min. The cells were then re-suspended in 10 ml of cold sterile 100 mM CaCl₂. Next, the cells were kept on ice for 30 min and then collected by centrifuging. Cells were resuspended in 100 ml of CaCl₂ containing 15% glycerol. The competent cells were then stored at −80° C. until needed.

Ligation Reaction

The ligation reaction was carried out by mixing the DNA vector fragment (phagemid) (0.020 pmol), insertion fragment from the PCR (0.060 pmol), 5× ligation buffer (4 pI), T4 ligase from Invitrogen (2 μl), and ddH₂O (8μ). The reaction was done at 25° C. for 2 hours.

Transfection of DNA into Competent E. coli TG1 Cells (CaCl₂ Method)

In a sterile 1.5 ml Eppendorf tube, 100 pI of competent cells and 20 pI of ligation mixture was mixed. The mixture was held in ice water for 1 hour, 90 sec at 42° C., and then 2 min in ice water. Luria broth medium (1 ml) was added to the tube followed by incubation at 37° C. for 1 hour in a shaking incubator. The transfected bacteria (200 pI) was then transferred onto a luria broth plate containing the respective antibiotics. The vector fragments without ligation were used as a control.

Confirmation of Recombinant Phagemid

Colonies of bacteria were then inoculated into luria broth medium and amplified. The phagemid was purified from the amplified bacteria and sent for sequencing (MCLAB) to confirm the insertion fragment was added and the sequence did not have mutations. It should be noted that the cell line TG1 was very poor for amplifying phages. This method was repeated using ER2738 competent cells (sequenced again) and the amplification of phages was greatly improved.

Amplification of Genetically Engineered M13 Phage

ER2738 (with recombinant plasmid) was inoculated into 5 ml of luria broth with 5 ul of chloramphenicol (50 mg/ml). The culture was incubated at 37° C. and shaken at 200 rpm overnight. 300 ul of overnight product was mixed with 1200 ul of luria broth with 45 ul of helper phage (M13KO7). The mixture was incubated without shaking at 37° C. for 1 hour, then transferred to 30 ml of luria broth. The mixture was then shaken for 1 hour followed by the addition of 30 ul of chloramphenicol additional shaking for 3 more hours. The mixture was then transferred to 2.0 L of lura broth. 2.0 ml of kanamycin (70 mg/ml) and 200 μl of IPTG (1 M) were added to the mixture. The mixture was then incubated for 24 hours at 37° C. with vigorous shaking. The mixture was then centrifuged at 8200 g for 50 min. The supernatant was transferred to a new container and mixed with 1/6 volume 20% PEG/2.5 M NaCl. The mixture was allowed to precipitate overnight at 4° C. Next, the mixture was centrifuged at 8200 g for 50 minutes at 4° C. The supernatant was discarded. The mixture was re-spun briefly followed by the removal of any residual supernatant. The pellet of phages was then resuspended in 10 ml of distilled water and transferred to a centrifuge tube. The mixture was then allowed to disperse more evenly over time by shaking it for 1 hour at room temperature. The mixture was then centrifuged at 7200 rpm for 50 minutes to remove any residual bacteria or other insoluble material. The supernatant was then transferred to a new tube and mixed with 1/6 volume 20% PEG/2.5 M NaCl. The mixture was then allowed to precipitate at 4° C. overnight. Next the precipitated mixture was centrifuged at 7200 rpm for 50 minutes. The supernatant was discarded, and the pellet was resuspended in 1 ml of distilled water. The solution was transferred to an Eppendorf tube followed by shaking for 1 hour at 200 rpm (or until the phage pellet fully dissolved). The solution was then centrifuged at 12,000 rpm on a table top centrifuge for 10 minutes to remove any insoluble material. The supernatant was transferred to a new tube and the optical density at 269 nm was measured to determine the concentration based on a Beer's Law concentration curve (optical density×1×10¹⁴=number of phages/ml). This process only works within the linear portion of the Beer's Law curve (from 0.1 to 1.0 optical density). To further purify the phages, the phages were then transferred into dialysis tubing. Dialysis was allowed to occur in 5 L of distilled water with water changes every 3 hours for 9 hours. A final water change was allowed to go overnight. Next, the phages were transferred to vortex tubes and vortexed for 1 minute. The phages were then passed through a 0.2 μm filter. The concentration could then be re-measured using the same optical density procedure.

Generation of Phage Films

This procedure can be scaled for any size well plates or petri dish, but the volumes included are for 96-well tissue culture plates. For each film, 120 μl of poly-L-lysine was pipetted into a well of a tissue culture 96-well plate. The solution was allowed to incubate at room temperature for 30 minutes. The poly-L-lysine was pipetted out and saved in a clean container. The plates were allowed to completely dry in a clean hood with the fan on. 100 μl of 1.5×10¹³ phage/ml was added to each well. The phage solution was allowed to incubate at room temperature for 30 minutes. The phages were transferred to a new clean container and the 96-well plate was dried in the hood again. 100 μl of the recycled poly-L-lysine solution was pipetted into each well and allowed to incubate at room temperature for 30 minutes. The poly-L-lysine solution was discarded, and the wells were allowed to completely dry again in the hood. 80 μl of the recycled phage solution was pipetted into each well. The coating was evenly spread across the wells. The solution was allowed to dry overnight in the hood. The films were exposed to UV light in a clean environment for 24 hours. The films were now complete. The films were then soaked in cell medium overnight before seeding any cells.

Characterization of M13 Phage by AFM and TEM

M13 phages were characterized by atomic force microscopy (AFM) on a BIOSCOPE catalyst with ScanAsyst AFM (Bruker). The samples were prepared by pipetting 40 μl of 3×10¹² phages/ml onto a glass slide followed by spin drying. The samples were imaged under 4 modes, height, inphase, peak force error, and quadrature. For transmission electron microscopy (TEM), 10 μl of 1×10¹² phages/ml were pipetted onto a formvar-coated TEM grid and allowed to soak for 3 minutes. The extra liquid was then wicked away from the grid using a piece of filter paper. 10 μl of 0.5% uranyl acetate at a pH of 4.5 was then added to the grid for 10 sec. The extra uranyl acetate was then wicked away. The grid was then blown dry quickly with a hair dryer. Images were then taken on a Zeiss 10 TEM at a magnification of 6300×.

Characterizing Phage Film Morphology

Phage films were generated according to the described protocol. The phage films were then imaged using a Nikoneclipse Ti microscope under brightfield imaging. Additionally, the phage films were imaged using a BIOSCOPE catalyst with ScanAsyst AFM (Bruker) under 4 modes, height, inphase, peak force error, and quadrature.

Cell Proliferation on Phage Films

hMSCs were seeded onto 96-well plate phage films (200 μl of 1×10⁴ cells/mL) and grown for 47 hours or 71 hours. Both stimulated and unstimulated controls were included. At the end of the indicated growth times, AlamarBlue® was added as 10% of the volume in the well mixed with cell media. The cultures were allowed to incubate 8 hours. The absorbance at 570 nm and 600 nm were then measured. The percent reduction of AlamarBlue® was then calculated (higher percent reduction=more growth).

Immunofluorescence of Osteoblast Marker Proteins

After culturing hMSCs on phage films for 26 days, the cells were fixed with 4% paraformaldehyde (10 min) and permeabilized with 0.2% Triton X-100 (2 min) at room temperature. Five washes with PBS were done after each step. The cells were then blocked with 5% goat serum solution for 1 h followed by 5 washes with PBS. The osteogenic protein markers osteopontin (OPN), osteocalcin (OCN), and collagen type 1A (COL) were detected through immunofluorescence. OPN was detected with the primary antibody for OPN (RbpAB to osteopontin ab8448 from Abcam). The secondary antibody used was Goat Anti-Rabbit IgG H&L (Alexa Fluor 555) (ab150078) from Abcam (red). OCN was detected with the primary antibody for OCN [OC4-30] (ab13418) from Abcam). The secondary antibody used was Goat Anti-Mouse IgG H&L (Dylight 650) pre-adsorbed from abcam ab96882 (red). COL was detected using a primary antibody for COL (RbpAb to collagen 1 ab34710). The secondary antibody used was Goat Anti-Rabbit IgG H&L (Alexa Fluor 555) (ab150078) from abcam (red). Cell nuclei were stained by DAPI (blue) for all samples, and actin was stained by phalloidin (green). The positive control was osteogenic media from Lonza ((hMSC Osteogenic Bullet Kit). All other samples were grown in basal media (MSCGM Bullet Kit (PT-3238 & PT-4105) from Lonza.

Calcium Nodule Staining

After 26 days of culturing hMSCs under differentiating conditions, the cell media was removed from the 6-well tissue culture plates. The plates were rinsed once with PBS. The cells were then fixed with 4% paraformaldehyde solution for 10 minutes. After fixation, the wells were rinsed twice with distilled water and the cells were stained with 2% Alizarin Red solution (pH 4.2) for 3 minutes. The wells were then rinsed 5 times with PBS, and the cells were imaged.

qPCR of Osteoblast Marker Genes

hMSCs were seeded (1×10³ cells per culture plate) on to phage films as well as relevant control plates. The cells were then cultured for 26 days. The cells then were processed with a Power SYBR™ Green Cells-to-CT™ Kit. The number of cells was adjusted to be 5×10³ cells per lysis reaction. All other parameters were kept the same as the manufacturer's protocol for this kit. The gene expression of osteopontin, osteocalcin, and collagen type 1A were measured as a fold change normalized to the reference gene GAPDH and a negative control (basal media+ cells but no phage) such that the baseline at zero-fold change represents the negative control. For temperature cycling and measuring CT values, a MiniOpticon Real Time PCR System was used.

Results

Sequencing Results of Phage Display

On the first sequencing of phage plasmid in TG1 bacteria, the results came out with the correct inserts. However, the amplification of engineered phages was poor in this strain of bacteria. Therefore, ER2738 competent cells were transfected with the plasmid instead. All sequences were displayed as desired (data not shown).

Organized Ridge Groove Structure of Phage Films

It is well known that M13 phages can self-assemble into ordered structures during liquid crystalline phase transitions. However, the consistency of these assembly strategies is far less than desirable as is often the case with M13 phage film structures. Due to repeatability issues, a new protocol was developed as described herein. This protocol involved alternating layers of M13 phages and poly-L-lysine for 2 layers of each. A major difference to reported protocols is that the M13 phages were both desalted and filtered through a 0.2 μm filter. This provided more clean and consistent structures. Also, in the final step the M13 phage solution was allowed to slowly dry overnight rather than pulling it off again. It was also very important to use fresh poly-L-lysine as well as good tissue culture plates. The resulting structures are highly organized and more repeatable (success 50% of the time on the entire plate, but large regions always had this structure even if the whole plate was not uniform). In addition, these phage films were highly durable supporting hMSC growth over the course of a month with no loss of structure. Additionally, hMSCs grown on these films align and stretch with the background structure of organized phages. Due to these properties, these films demonstrated improvement over previously published methods.

Phage Films Support hMSC Growth and Proliferation

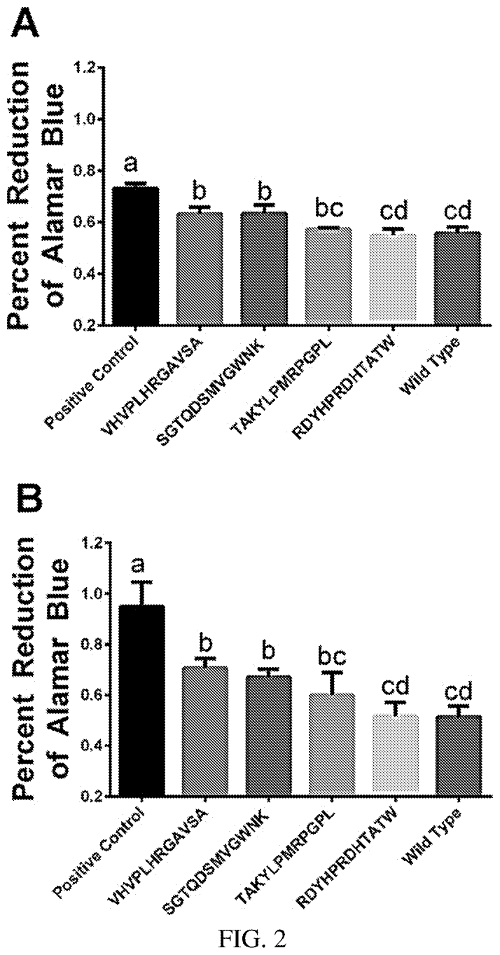

The phage films did support stem cell growth and proliferation as seen in FIG. 2. hMSCs on the phage films did proliferate to densely cover the entire plate as was seen in later differentiation studies.

Peptide-Induced Differentiation of hMSCs

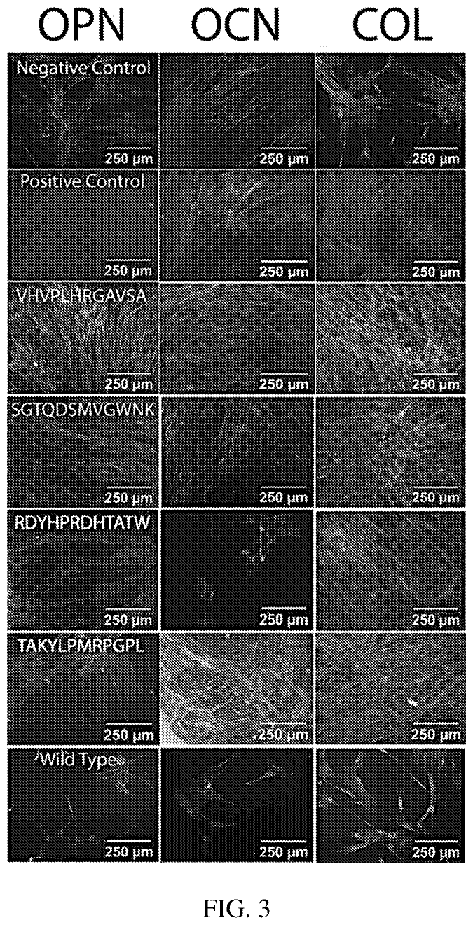

The peptide-phage films induced differentiation of hMSCs as demonstrated by production of osteoblast marker proteins. The osteoblast marker proteins osteopontin (OPN) and osteocalcin (OCN) were detected using immunofluorescence. Collagen type 1A was used as a control which shows the same level of expression on each different peptide-bearing phage film. The results are shown in FIG. 3. All peptides on phage films showed expression of osteopontin including the wild type phage film. This indicates that the highly ordered ridge groove structure of the phage films can induce osteoblast differentiation. Additionally, the peptide VHVPLHRGAVSA showed a qualitatively higher level of osteopontin protein expression than the other groups and a higher expression than the positive control (osteogenic media from Lonza). For osteocalcin, the expression levels were low on every sample including the positive control, but again VHVPLHRGAVSA appeared to produce slightly greater osteocalcin production. These data demonstrated that the phage films could induce osteogenesis, with the VHVPLHRGAVSA peptide sequence showing greater results. The other sequences also showed differentiation but required more quantitative results to demonstrate positive results than the qualitative immunofluorescence images.

Peptide-Phage Induced hMSC Calcium Deposits

Calcium nodule staining was done using Alizarin red at 14 and 26 days. At 14 days, hMSCs on the phage films and those cultured in osteogenic media showed an increase in calcium deposits at similar levels. In this test, it was not possible to establish a difference between osteoblast induction through the topography of the phage films vs from the peptides used on each phage film. However, it did show that each phage film after 14 days was able to induce calcium deposits on the level of the positive control which is a leading product for osteogenesis. At 26 days, a major difference between the negative control, phage film samples, and the osteogenic media was observed. All phage films, although indistinguishable from each other by this method, performed substantially better than the negative or positive control. This indicated that the hMSCs deposited a mineralized matrix which is indicative of osteoblasts when cultured on phage films. Additionally, the phage films may be an alternative to using osteogenic media as they performed better than the positive control.

Peptide-Phage Induced Genetic Upregulation of Osteoblast Marker Genes

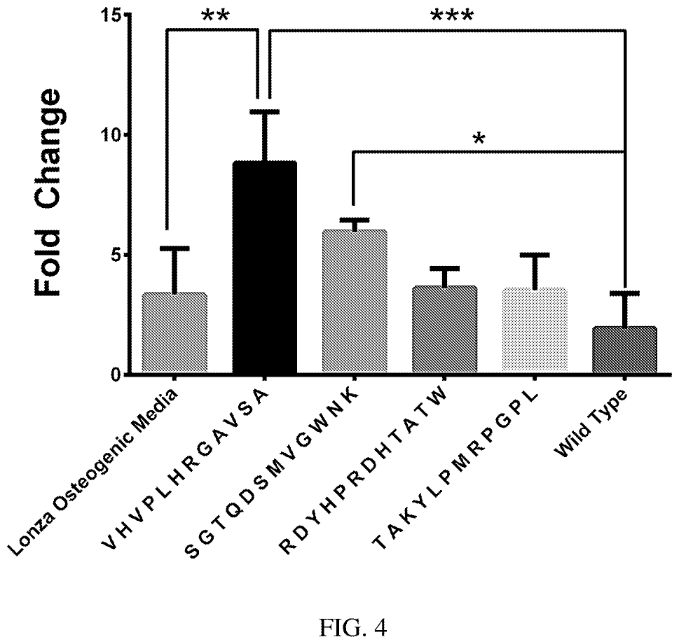

Higher osteopontin expression is an indicator of osteoblast differentiation. FIG. 4 shows results of osteopontin expression of hMSCs cultured on phage films or under control conditions after 26 days. All phage film conditions and the positive control (osteogenic media from Lonza) showed an increase in osteopontin expression relative to the negative control represented by the baseline. The negative control was hMSCs cultured in basal medium for 26 days. The peptides VHVPLHRGAVSA and SGTQDSMVGWNK demonstrated a statistically significant advantage over the wild type phage films. Additionally, VHVPLHRGAVSA showed a statistically significant advantage over the positive control which is an established leading company product. These results demonstrate that at least the two peptides VHVPLHRGAVSA and SGTQDSMVGWNK are potent osteoblast inducers in regard to osteopontin gene expression. Taken together with the previous immunofluorescence results, these peptides upregulate osteopontin at both the protein and mRNA levels. Lastly, this data demonstrates sustained osteopontin upregulation at the later stages of osteoblast differentiation.

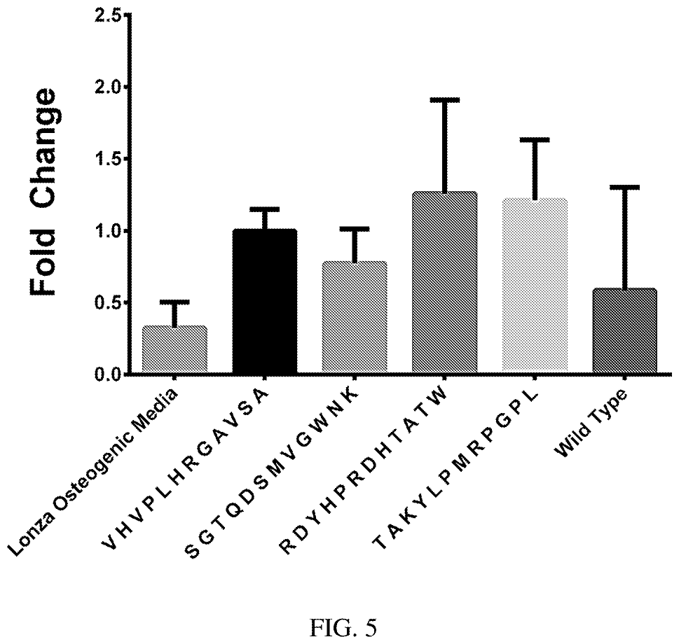

The final mineralized extracellular matrix created by osteoblasts is primarily composed of collagen type 1 along with smaller amounts of osteocalcin, matrix gla protein, osteopontin, bone sialoprotein, bone morphogenic proteins, TGF-β, and hydroxyapatite. Therefore, collagen type 1a gene expression was measured (FIG. 5). The expression levels for this gene were expected to be constant. From the qPCR assay, the collagen type 1a expression appeared slightly raised in samples with phage films, but no significant difference was detected. Comparing the qPCR fold-change data to the immunofluorescence data, the expression of collagen type 1a appears to remain consistently high without any changes due to the peptides, phage films, or positive controls used.

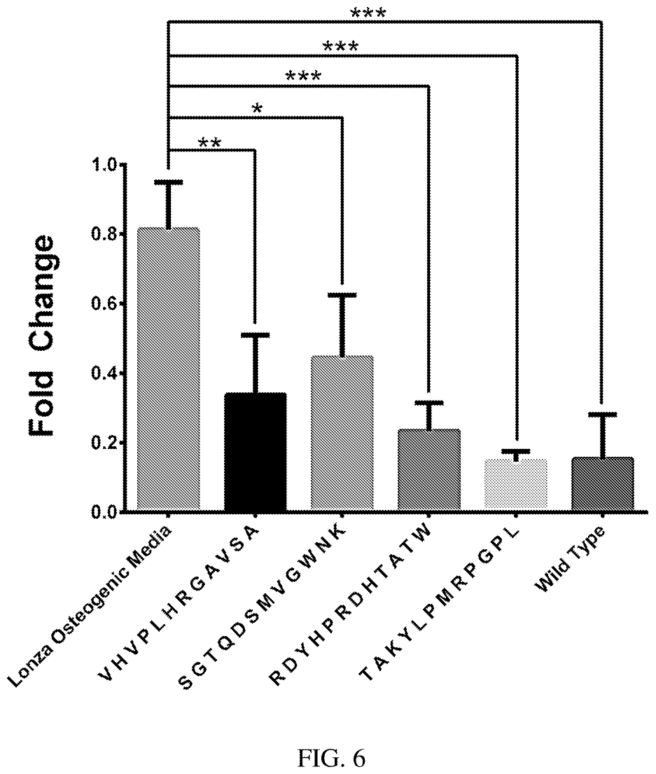

Osteocalcin is an indicator of osteoblast differentiation. As shown in FIG. 6, all peptide-bearing phage films and the positive control (osteogenic media) showed fold changes in osteocalcin production above the baseline represented by the negative control (hMSCs cultured in basal medium). The positive control (osteogenic media from Lonza), showed a small but statistically different advantage relative to the peptide-bearing phage films for osteocalcin production. However, the fold change values for osteocalcin were relatively low. Comparing the qPCR data to the immunofluorescence data, osteocalcin appears to be produced in only small amounts at both the protein and mRNA levels for all differentiation conditions.

In summary, the morphology of M13 phages was characterized by TEM and AFM. The M13 phages amplified were demonstrated to be filamentous in structure measuring ˜1000 nm in length. The phages also demonstrated a self-assembly into highly ordered and aligned bundling structures brought about during a liquid crystalline phase transition. The natural ability of these phages to self-assemble into ordered structures was then used to develop a new, more reliable phage film generation strategy. This strategy produced highly organized phage films with an aligned ridge groove morphology. This morphology was shown to be a potent biophysical cue for osteoblast differentiation at a genetic level, protein production level, and demonstrated large amounts of calcium deposition (greater than a leading company product). The novel peptides identified in Example 1 were then genetically displayed on the pVIII coat protein of M13 phages. When the peptide-bearing phage films were assembled, they allowed for a side-by-side comparison of the effects of each peptide on differentiation relative to the wild type phage control. The peptides, particularly VHVPLHRGAVSA and SGTQDSMVGWNK, demonstrated abilities to induce osteoblast differentiation as indicated by osteoblast marker proteins, marker genes, and calcium mineral deposits. The peptide VHVPLHRGAVSA demonstrated an ability to induce differentiation of hMSCs better than a leading company product, osteogenic media from Lonza. These results demonstrated that these newly discovered peptides are in fact osteoblast differentiation inducing peptides. Their effectiveness in stimulating bone regeneration was further examined in the following Example.

### Example 3

Development of Bone Regenerative Materials

Personalized 3D Printed Bone Regenerative Scaffolds for Implantation

3D printing is an advantageous technique for bone defect repair as it can be customized to a patient's unique bone defect. Micro-computed tomography (Micro-CT) can be used to image the patient's bone defect and generate a 3D computer model. From this, scaffolds can be printed to exactly match the defect to establish a personalized therapeutic approach to bone repair. Compared to alternate 3D-printed bone materials, titanium alloys have the major advantage of being mechanically strong enough for load bearing sites. Titanium alloys are also inherently bioinert, making them an ideal implantable material.

There are several major issues in the field of bone implants which can be improved upon. Bone implants need to closely mimic the native extracellular matrix (ECM) which can lead to improved cell adhesion, differentiation, proliferation, and tissue regeneration. These qualities are essential for the bone implant to be in harmony with the native environment, ensuring that the defect area can be replaced with functional, healthy tissue having little or no reparative scar formation. Additionally, microscale features that allow for cell penetration, nutrients diffusion, vascularization, and spatial organization of cellular growth are essential. These qualities can improve upon vascularization limitations by allowing the diffusion of nutrients and possibly inducing vascularization. The requirements have been achieved herein. After creating these scaffolds, they could be modified with the presently disclosed osteoblast differentiation-inducing peptides and seeded with hMSCs. The peptides can induce hMSCs to differentiate into osteoblasts and thus further improve bone regeneration. The osteoblasts can then generate new bone tissue and differentiate into osteocytes when encased by bone matrix secretions and offer long-term maintenance of bone.

Freeze Casting Organized Bone-Mimetic Lamellar Structures

Freeze casting is a process in which the directional growth of ice crystals can push solid materials dissolved in an aqueous solution into an organized pattern. Upon being completely frozen, the ice can be removed through sublimation during which the sample is put under vacuum. When the directionality of the ice crystal growth is controlled by precise manipulations of the temperatures on all sides of the liquid suspension, complex, ordered structures can be formed. When ice crystals are either grown directionally from the bottom up, top down, or a combination of these two, a lamellar structure can be obtained. A lamellar structure closely mimics the natural structure of bone. Thus, such lamellar structures are preferred to help provide a biophysical environmental cue for bone regeneration. The following non-limiting examples and experiments were performed to demonstrate the novel scaffolds and uses thereof disclosed herein.

Methods

Various chemicals and materials used herein included calcium nitrate tetrahydrate, carbon dioxide (CO₂) gas, ethanol, 1-ethyl-3-(3 dimethylaminopropyl) carbodiimide hydrochloride (EDC), hydroxyapatite (HAP), isoflurane, Lidocaine, linoleic acid, octadecylamine, oxygen gas, paraformaldehyde, polyethylene glycol-8000 (PEG 8000), sodium hydroxide, and sodium phosphate,

3D Printing Titanium Alloy

3D printed titanium implants were created using a biocompatible metal alloy made of titanium, aluminum, and vanadium (Ti-6Al-4V, 6:4:90, Al:V:Ti, wt %) which is among the most commonly used titanium alloys for biomedical implants. Other alloy compositions described elsewhere herein can be used instead. Specifically, a design of the desired porous scaffold was created in the digital format known as an STL (Stereo Lithography) file using a computer-aided design (CAD) software. Then commercial Ti-6Al-4V alloy powder (Sandvik, Sweden) was printed into an implant in pure Ar gas according to the STL file, by means of selective laser melting (SLM) through a commercial laser-based 3D printer (Concept LASER M2) specialized for printing metallic powder. The 3D printer was equipped with a Yb-FaserLaser with a focus beam diameter of 50 μm. The SLM parameters during 3D printing included a laser power of 100 W, a scan speed of 650 mm/s, a scan spacing of 70 μm, and a layer thickness of 30 μm. The scaffolds were cylindrical in shape with a height of 8 mm and a diameter of 5 mm. The pores within the scaffold measured ˜200 μm in diameter.

Creating a Double-Sided Freeze Casting Machine.

A double-sided freeze casting machine to form the scaffolds was built loosely based on a published design (Waschkies, T.; Oberacker, R.; Hoffmann, M. J., Investigation of structure formation during freeze-casting from very slow to very fast solidification velocities. Acta Mater. 2011, 59 (13), 5135-5145). A double-sided freeze casting system allows for greater control over generating constant solidification velocities and can produce nearly constant lamellae spacing over a longer sample height relative to a one-sided freeze casting system. Two Omega temperature controllers were integrated into the double sided freeze casting system to allow for a temperature control system. The controllers were programmed to either be able to allow for drops of 3° C./min for both the top and bottom copper rods or for a drop of 3° C./min for just the bottom rod while maintaining the top rod at 0° C. (typically done unless otherwise stated). The final temperature was 135° C. Reservoir of liquid nitrogen were held in contact with the top and bottom copper rods to provide a cold source. Any liquid suspension to be freeze casted could fit in various sizes of containers which all fit in between the two copper rods. The containers included top and bottom copper pieces which perfectly fit against the copper rods and a plastic cylindrical container that goes between them. The cylindrical container was open on both ends.

HAP Nanorod Synthesis, TEM Imaging of HAP, and Powder X-Ray Diffraction of HAP.

Hydrophobic hydroxyapatite (HAP) nanorods were synthesized according to a published protocol (X., W.; J., Z.; Q., P.; D., L. Y., Liquid-Solid-Solution Synthesis of Biomedical Hydroxyapatite Nanorods. Adv Mater 2006, 18 (15), 2031-2034). After synthesizing, the HAP nanorods were pelleted by centrifugation and washed with ethanol, pelleted in a centrifuge again, resuspended in water, frozen at −80° C., and freeze dried overnight. The nanorods were then ball milled overnight to obtain a fine HAP powder. For TEM imaging, 10 μL HAP nanorods suspended in water were pipetted onto a formvar-coated TEM grid. After 3 min, any extra liquid was wicked away using a filter paper and the grid was dried using a hair dryer. The HAP nanorods were then imaged on a Jeol 2000 TEM at 200 KV and 100 K Magnification. Additionally, X-Ray diffraction was done on the ball-milled HAP powder.

Generating Bone Mimetic Lamellar Structures of Combination of Phage, Hydroxyapatite, and Collagen Through Freeze Casting, Cross Linking, and Sintering Processes

Various solutions for freeze casting were optimized and their components are listed in Table 3. Each solution was freeze-casted using the constructed double-sided freeze casting machine with a temperature drop of 3° C./minute and a final temperature of −135° C. The frozen solutions were then freeze dried overnight. Images of the unprocessed scaffolds were taken by SEM after sputter coating with gold/palladium. Additional processes were also done to strengthen composite scaffolds.

TABLE 3
Biodegradable freeze casting recipes.

| | | Rat tail | Hydroxy- | |
|---|---|---|---|---|
| | | collagen | apatite | |
| Type of | M13 phage | type 1 | nanorod | |
| scaffold | Amount | amount | amount | PEG-8000 |
| Pure Phage | 1 mL of | None | None | None |
| | 9 × 10¹³ | | | |
| | phages/mL | | | |
| Phage + HAP + | 1 mL of | 100 μL of | 25 mg | None |
| Collagen | 9 × 10¹³ | 9.87 mg/mL | | |
| | phages/mL | | | |
| HAP | None | None | 50 mg in | 20.8 mg |
| | | | 1 mL of H₂O | |

For the phage+collagen+HAP scaffold, crosslinking with 1-ethyl-3-(3 dimethylaminopropyl) carbodiimide hydrochloride (EDC) was done. Briefly, the scaffold was dropped into a solution of 200 mM EDC in ethanol. After incubating for 12 hours, the scaffold was placed in 0.1 M sodium phosphate for 2 hours to hydrolyze any unreacted o-isoacylurea intermediate. The scaffold was then soaked in PBS (pH 7.4) for 2 hours. To image this scaffold, the scaffold was dehydrated in a series of ethanol solutions (20%, 50%, 70%, 80%, 90%, 95%, 100%) incubating at each step for 20 minutes followed by critical point drying. The scaffold was then sputter coated with gold/palladium before SEM imaging. For the pure HAP scaffolds, sintering was done after the freeze-drying process. For the first phase of sintering, the temperature was raised from 25° C. to 550° C. linearly over a time span of 2 hours and 55 minutes. The temperature was then held at 550° C. for 2 hours. The temperature was then raised to 950° C. linearly over 2 hours and 13 minutes. The temperature was held at 950° C. for 5 minutes followed by linear cooling to 25° C. over 5 hours and 8 min. For imaging, the scaffold was then gold/palladium sputter coated and imaged by SEM.

Freeze Casting M13 Phages onto 3D Printed Titanium

The titanium alloy scaffolds were rinsed 3 times in distilled water followed by autoclaving. The scaffolds were then coated in poly-L-lysine by soaking in 2.5 ml of poly-L-lysine per 5 scaffolds in a 24-well cell culture plate for 30 minutes. The poly-L-lysine was then discarded, and the scaffolds were allowed to dry in a sterile hood overnight. In a sterile cell culture hood, 2 scaffolds were loaded into the sterile freeze casting holder (sterilized by autoclaving). 2 ml of sterile, filtered phage solution (3×10¹³ phages/ml) was pipetted into the casting holder and directly into the pores of the titanium scaffolds. The solution was allowed to incubate at room temperature for 30 minutes so that phages could stick better to the scaffold. The scaffolds and phage solution were then freeze casted in the double-sided freeze casting machine with a temperature drop of 3° C./min on the lower contact point and a constant 0° C. on the upper contact point. The final temperature for the lower contact point was −135° C. The frozen scaffold and solution were then freeze dried overnight. For cell seeding, the scaffold was then soaked overnight in cell medium followed by seeding 3.6×10⁴ hMSCs onto the scaffold the next day in new medium. After 1 day of culturing, the scaffolds were implanted into the rat radial bone defect model.

Scanning Electron Microscopy (SEM), EDS, and Cell Attachment to Scaffolds

After the freeze casting or cell seeding protocols for the titanium scaffolds, the scaffolds were fixed in 4% paraformaldehyde solution for 10 min. The scaffolds were then washed 5 times in PBS (pH 7.4). The scaffolds were then dehydrated in a series of ethanol dilutions (20%, 50%, 70%, 80%, 90%, 95%, 100%) incubating at each step for 20 minutes followed by critical point drying. The scaffold was then sputter coated with gold/palladium before SEM or energy-dispersive X-ray spectroscopy (EDS) imaging.

Mechanical Strength Testing

Compression tests were done to determine the mechanical properties of the titanium scaffolds. The tests were performed using a Test Resources mechanical tester (800LE4AT30). The load cell value was 12.5 kN at room temperature. The cross-head speed was set to 0.008 mm/min. The elastic modulus was calculated from the linear region of the stress-strain curve using the 0.2% offset method. For the healthy bone samples, a three-point bending test was done. The tests were also carried out using a Test Resources mechanical tester (800LE4AT30). The load cell value was 12.5 kN and the cross-head speed was set to 0.005 mm/min. The cross-head speed was set to 0.008 mm/min. The elastic modulus was calculated from the linear region of the stress-strain curve using the 0.2% offset method.

Rat Radial Bone Defect Model

A rat radial bone defect model was generated for evaluating the bone regeneration potentials of these 3D printed titanium scaffolds. The defect was created by removing an 8 mm section of the radius bone. The animals were anesthetized by inhalation of 4% isoflurane/oxygen (v/v) mixture using an anesthesia machine (VT-110 small animal anesthesia machine) and then with 2% isoflurane (v/v) during the surgical operation with a non-rebreathing system. A radial defect of 8 mm was created using a rongeur unit (bone cutter). The defect was thoroughly rinsed with isotonic saline and any bone fragments were removed. After implanting the scaffold, the incision was closed with 5-0 sized silk sutures. A subcutaneous injection of 2% Lidocaine (w/v) was administered immediately directly into the surgical area under the skin after closure of the incision to relieve pain. At the end of the surgery, the animal was administered a subcutaneous injection of sterile saline at a dose of 10 ml/kg per surgery hour and provided pure oxygen by a non-rebreathing system until it awakened from the isoflurane anesthesia. A dose of carprofen (anti-inflammatory drug) solution was subcutaneously given (4 mg/kg) every 12 hours for 7 days.

Preparation and Fixation of Harvested Bone Defect Tissues

After 4 weeks and 12 weeks, animals (N=5) were euthanized by CO₂ asphyxiation. The sections of the arms containing the implant (at the radius bone) were then cut way from the rest of the animal using bone cutters. The 4-week implant samples were fixed in 10% (w/v) neutral formalin solution for 4 days. The 12-week samples were fixed in 4% paraformaldehyde for 4 days. Paraformaldehyde was used as it can produce better CD31 vascularization stains relative to fixing with formalin. The samples were then placed in a solution of 70% ethanol to 30% water (v/v).

Hematoxylin and Eosin Tissue Staining

The tissue sections were deparaffinized and hydrated with distilled water. Hematoxylin was applied to the sample with enough volume to cover the tissue section followed by incubation for 5 min. The mounted tissue slide was then rinsed in 2 changes of distilled water (sections were allowed to soak for 15 sec on each exchange of water). Enough Bluing Reagent was then added to the tissue section to completely cover it followed by incubation for 10-15 sec. The slides were then rinsed again in 2 changes of distilled water allowing 15 sec of contact with the water each time. The slides were then dipped in 100% ethanol for 10 sec. The excess ethanol was blotted off. Enough Eosin Y Solution was then added to completely cover the tissue section followed by incubation for 2-3 min. The slides were then rinsed in 100% ethanol for 10 sec. Next, the slides were dehydrated in 3 changes of 100% ethanol allowing 1-2 min incubation times at each exchange of ethanol. The sections were then cleared and cover slipped.

Von Kossa Tissue Staining

The sections were deparaffinized and hydrated in distilled water. The slides were then incubated in Silver Nitrate Solution (5%) for 30-60 min while exposed to an ultraviolet light. The slides were then rinsed in 3 changes of distilled water. The slides were then incubated in Sodium Thiosulfate Solution (5%) for 2-3 min. The slides were then rinsed for 2 min in running tap water followed by two changes of distilled water. The slides were then incubated in Nuclear Fast Red Solution for a duration of 5 min Next, the slides were rinsed for 2 min in running tap water followed by 2 changes of distilled water. The samples were then dehydrated rapidly in 3 changes of fresh Absolute Alcohol. The slides were then cleared and mounted in synthetic resin.

Results

Freeze Casting Machine Performance

The double-sided freeze casting machine allowed for greater control over the linearity of the cooling rate of samples. Additionally, the top cold finger allows for control over the heat flux of the top of the sample. Another side benefit is that the sample can be completely sealed in a sterile container during the freeze casting when both a top and cold finger are used. Overall, the double-sided freeze casting system is superior to the single-sided system.

Hydroxyapatite Powder X-Ray Diffraction and TEM Confirms Synthesis of HAP Nanorods

Hydroxyapatite (HAP) nanorods were synthesized to be later used in freeze casting applications for bone regenerative scaffolds. X-ray diffraction was also done on the nanorods which confirmed that the nanorods were composed of hydroxyapatite.

Pure Phage and Phage+Hydroxyapatite Characterization after Freeze Casting

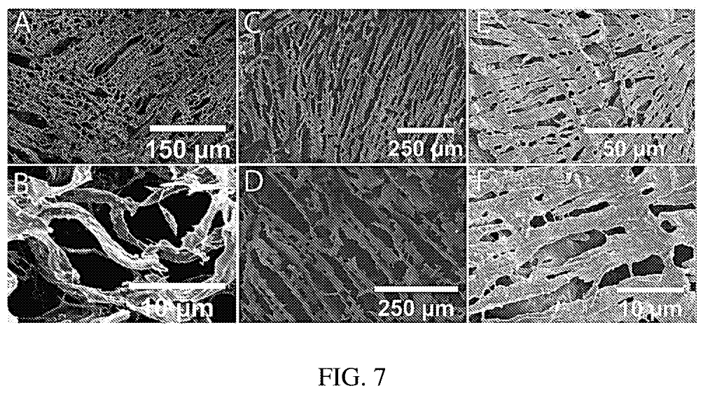

Using the double-sided freeze casting machine, several different lamellar scaffold structures could be created for their intended use in bone regeneration. FIG. 7 shows the resulting lamellar structures. A close up of the pure phage scaffold shows the fiber-like structure of M13 phages which form bundles within the scaffold (FIG. 7 A, B). The pure phage scaffold was highly organized into a lamellar structure. However, pure phage scaffolds are rather weak to handle and will fall apart if not moved very carefully. They are not ideal for load-bearing site bone regeneration. Additionally, the pure phage scaffolds will dissolve in water within minutes making them unsuitable for cell culture or cross-linking through EDC. However, when pure phage scaffolds are combined with HAP nanorods, the structure collapses and the scaffolds are too weak to visualize. Therefore, an additional component, rat tail collagen type 1 was added. Upon the addition of the collagen, the scaffolds were stronger and could be combined with HAP (FIG. 7 C, D). However, these scaffolds were still easily degraded if not handled carefully and their durability in water was only ˜2 days. Therefore, additional strengthening was necessary which was provided by crosslinking with EDC. The cross-linked samples are shown in FIG. 7 E, F. The crosslinked samples were stable in water or cell culture medium for greater than 2 weeks. However, their ability to regenerate bone in a load-bearing site was not possible as they had the consistency of a marshmallow. Due to the poor mechanical strength of this method, more mechanically strong methods were later pursued. However, this data does show that phages can be organized into a lamellar bone-like 3D structure using double-sided freeze casting. Additionally, upon strengthening the phages were semi-stable in water indicating some potential in vitro. These results were later used to develop a material coating method in which phages could be used to coat materials.

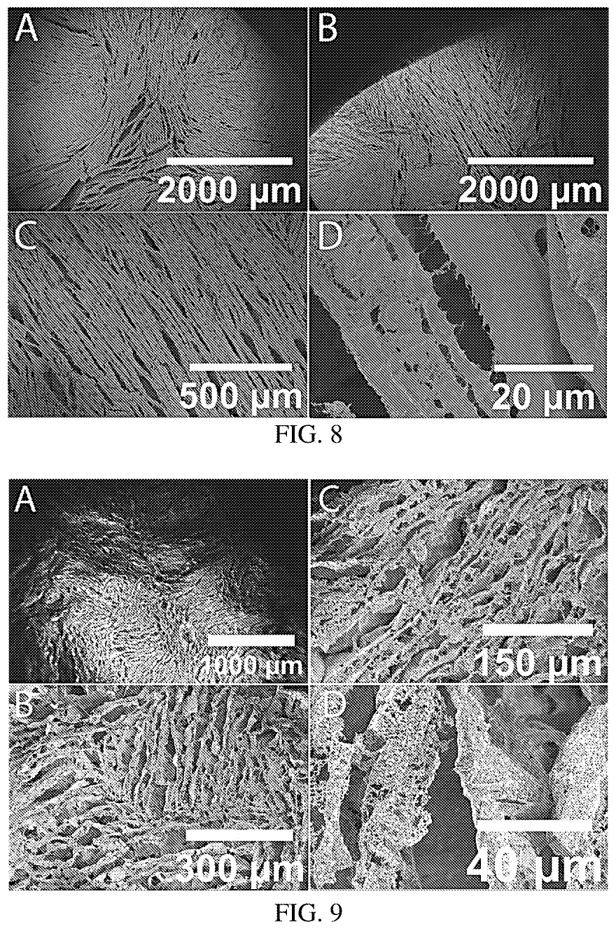

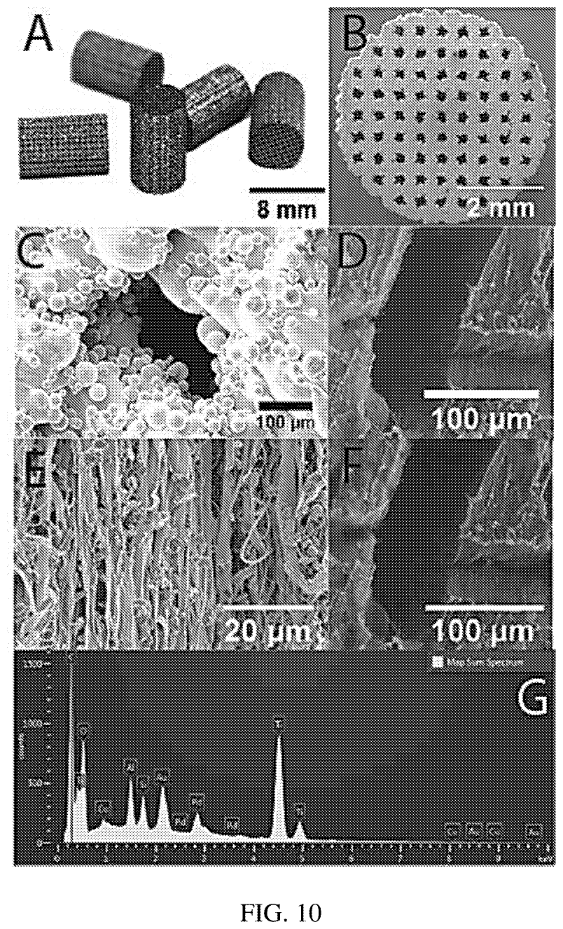

The double-sided freeze casting system was also used to generate HAP scaffolds held together with an organic binder, PEG-8000. These scaffolds showed excellent lamellar structures as shown in FIG. 8. However, these scaffolds also easily dissolved in water and had a stiffness comparable to a marshmallow. Therefore, the scaffolds were sintered. This process also served to burn off the PEG-8000 leaving only pure HAP left in the scaffolds resulting in the structures shown in FIG. 9. While the sintered HAP scaffold appears to be a bit shriveled relative to the un-sintered scaffold, the highly organized lamellar structure was maintained. Additionally, the scaffolds were now more durable and hard in nature. However, the scaffolds were still not considered strong enough for load-bearing sites as they could be broken with a very modest amount of pressure. This data does demonstrate the power of the double-sided freeze casting system for organizing bone regenerative materials into a lamellar structure which could have potential in vitro.

3D Printed Titanium Visual Characterization Before and after Freeze Casting or Cell Seeding

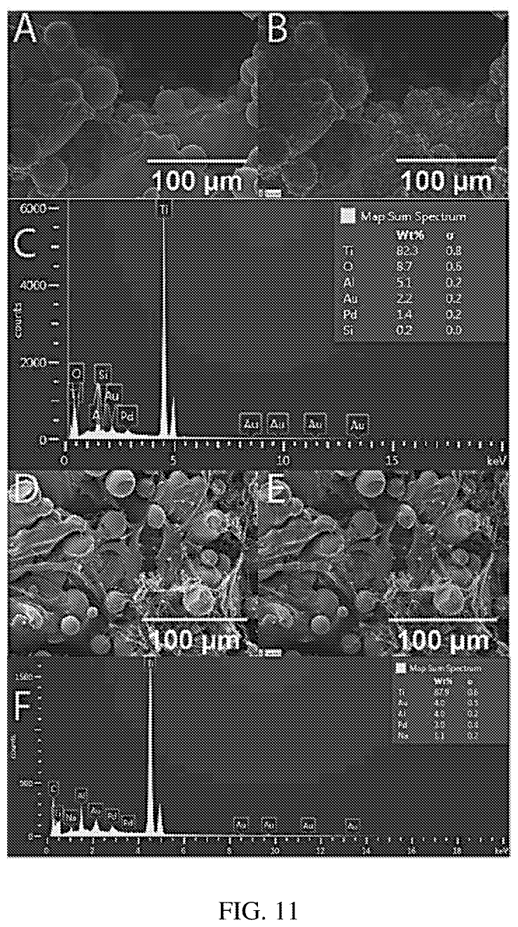

A stronger material for constructing the scaffolds was sought and titanium was investigated. Titanium scaffolds were constructed using 3D printed titanium scaffolds. Examples are shown in FIG. 10. The surfaces of the titanium on the sides were covered in several spherical titanium structures as a result of the printing method. M13 phages were freeze casted onto the titanium scaffolds, forming lamellar fiber-like structures along the scaffold, but the pores were still mostly accessible as shown. This lamellar structure closely resembles the natural structure of bone and was expected to provide an additional biophysical cue for increasing osteoblast differentiation. Additionally, the EDS mapping spectrum shows the composition of the phage coated scaffold to be mostly titanium, aluminum, and vanadium underneath a layer of phages (carbon). FIG. 11 shows that hMSCs can attach to and grow on the surface of both uncoated and phage-coated titanium scaffolds.

Mechanical Strength Properties of Titanium-Phage Hybrid Scaffolds Before and after Implantation In Vivo

Stress strain curves for 3D printed scaffolds and healthy bone samples were created. The average Young's modulus for titanium scaffolds not implanted was 3.7917±1.1284 GPa. The average Young's modulus for titanium scaffolds implanted for 4 weeks was 3.8436±0.7260 GPa. The average Young's modulus for healthy bone (4-week-old SD rats) was 10.1099±5.1366 GPa. No statistical difference was found between the Young's modulus for each group. The average maximum stress before failure of the titanium scaffolds not implanted, implanted 4 weeks, and healthy bone (4-week old SD rats) was 305.9027±14.2514 GPa, 278.1823±20.9649 GPa, and 229.1154±79.8224 GPa respectively. There was no statistical difference between the maximum stress before failure for the implanted scaffolds or healthy bone. This data demonstrates that the titanium alloy scaffolds are mechanically similar to healthy bone.

Titanium Scaffold-Bone Regrowth Assessed by Micro-CT

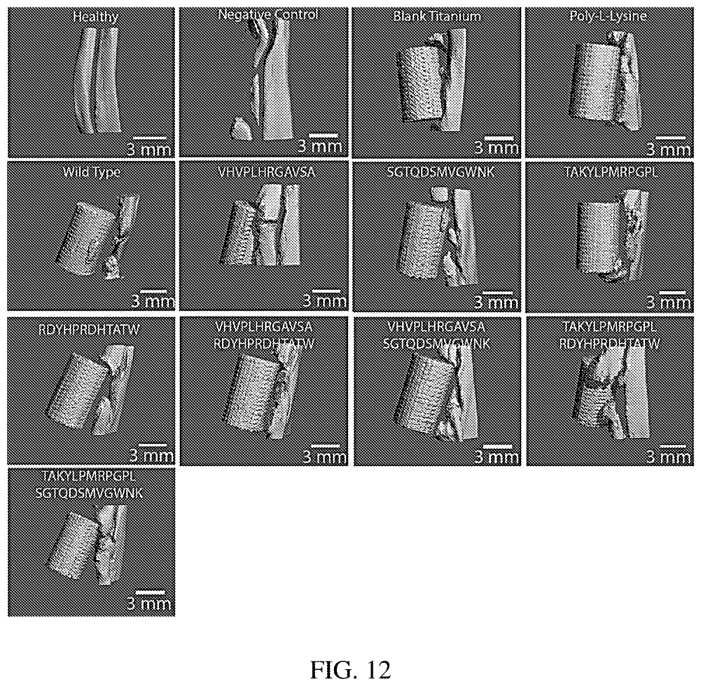

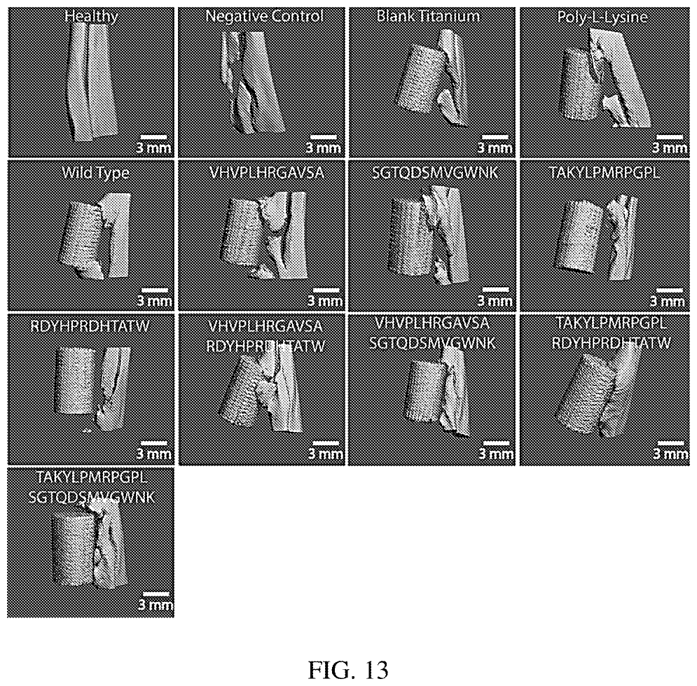

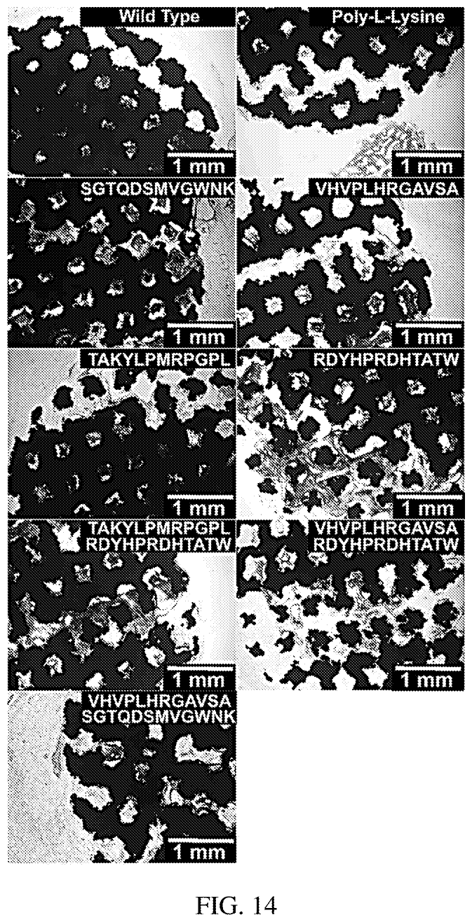

Titanium scaffolds were treated with phages displaying several osteogenic peptides including TAKYLPMRPGPL (SEQ ID NO:1), VHVPLHRGAVSA (SEQ ID NO:2), SGTQDSMVGWNK (SEQ ID NO:3), and RDYHPRDHTATW (SEQ ID NO:4), singly and in combination. FIG. 12 shows Micro-CT images of the various scaffolds implanted in the rat radial bone defect model. Images were taken 4 weeks after the defect was created. Increased healing was best observed in VHVPLHRGAVSA alone and a combination of TAKYLPMRPGPL+RDYHPRDHTATW. In a separate experiment, the Micro-CT images of scaffolds implanted in a rat radial bone defect model were taken 12 weeks after the defect was created (FIG. 13). Increased healing was observed in samples coated with more than one peptide, e.g., VHVPLHRGAVSA+RDYHPRDHTATW, VHVPLHRGAVSA+SGTQDSMVGWNK, TAKYLPMRPGPL+RDYHPRDHTATW, and TAKYLPMRPGPL+SGTQDSMVGWNK.

Histological Staining of Bone Regeneration Samples

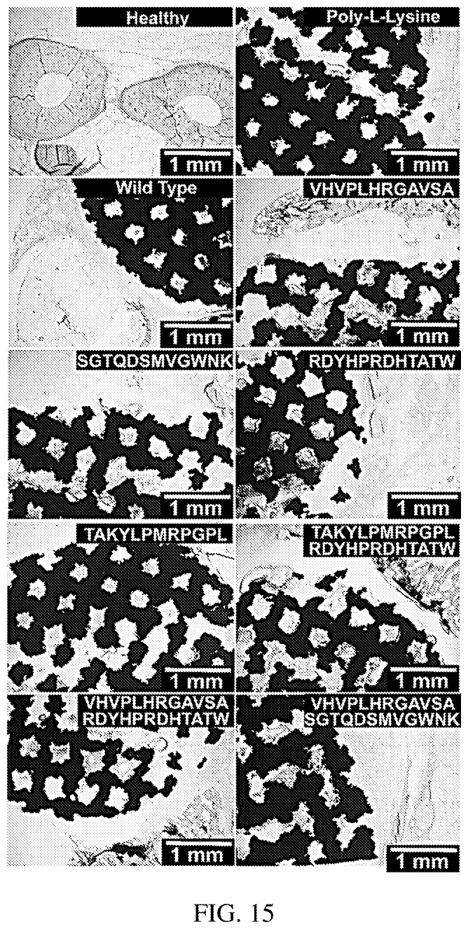

Samples of the implanted scaffolds of FIG. 12 were studied using histological staining. FIG. 14 shows top down views of HE-stained sections of each cylindrical-shaped implant. The dark black grids are the titanium and the cells can be seen in purple/pink. The scaffolds supported the penetration and growth of cells even to the deepest regions of the scaffolds. These implants were imaged after 4 weeks of implantation into the radial defect model. Cellular growth is visible in each of the scaffolds containing peptide-displaying phages. FIG. 15 shows top down views of Von Kossa-stained sections of each cylindrical-shaped implant. The dark black grids are the titanium. Other black areas are heavy calcium deposits. Gray areas are lighter calcium deposits. The lightly colored pink/purple are cells. New bone tissue was visible in all treated samples but was observed to be best around the scaffolds labeled VHVPLHRGAVSA and the combined TAKYLPMRPGPL+RDYHPRDHTATW.

In summary, as shown herein, phage-based scaffolds were created with lamellar structures resembling natural bone. However, the biodegradable phage scaffolds created from pure phage, phage and collagen, or additions of hydroxyapatite with phage while suitable for non-load-bearing repairs of bone tissue, were not suitable for load-bearing bone tissue repair due to insufficient mechanical strength. Titanium implants were created by 3D printing and were coated with phages genetically engineered to have osteogenic peptides, providing implants with mechanical strength much closer to that of natural bone. The phage coatings were aligned in lamellar structures through freeze casting onto the titanium. In a rat radial bone defect model, bone regeneration was discovered in the areas surrounding the implant. Cell penetration deep into the implants was observed through H&E staining. New calcium deposits were found within and around the scaffolds resembling natural bone via a Von Kossa stain. While these osteogenic peptide-coated implants were used herein in a radial bone defect model, the process can be used for coating any material, such as, but not limited to, dental screws or any hardware implanted and integrated with boney material.

In at least certain embodiments, the present disclosure is directed to a peptide conjugate having at least one peptide linked to a carrier molecule or a carrier nanoparticle, the at least one peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), TSKYLTMRPGPK (SEQ ID NO: 10), and variants thereof. The peptide may be linked to a linker molecule. The peptide may be displayed on a phage. The peptide may be linked to a film. The peptide may have human mesenchymal stem cell (hMSC) stimulatory activity via binding to a bone morphogenic protein receptor (BMPR). The peptide variant may have at least 75% identity to one of SEQ ID NO:2, SEQ ID NO:1, SEQ ID NO:3, SEQ ID NO:4, SEQ ID NO:5, and SEQ ID NO:6, SEQ ID NO:7, SEQ ID NO:8, SEQ ID NO:9, and SEQ ID NO: 10.

In at least certain embodiments, the present disclosure is directed to a peptide-implant complex, having at least one peptide linked to a surface of an implant, the at least one peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof. The peptide-implant complex may have a coating on the surface of the implant, the coating comprising the peptide. The implant of the peptide-implant complex may be a porous scaffold. The implant may be constructed of a material selected from the group consisting of natural cancellous bone, demineralized natural cancellous bone, collagen, calcium phosphate, hydroxyapatite, ceramic, polymer, metal, and metal alloy, and combinations thereof. The metal alloy of the bone implant may comprise titanium. The peptide-bone implant complex may be seeded with mesenchymal stem cells (MSCs).

In at least certain embodiments, the present disclosure is directed to an osteogenic bone-compatible matrix comprising a porous matrix infused with a peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof. The osteogenic bone-compatible matrix may be constructed of a material selected from the group consisting of natural cancellous bone, demineralized natural cancellous bone, collagen, calcium phosphate, hydroxyapatite, ceramic, polymer, metal, and metal alloy, and combinations thereof. The osteogenic bone-compatible matrix may be seeded with mesenchymal stem cells (MSCs).

In at least certain embodiments, the present disclosure is directed to a method of treating a bone defect in a subject in need of such treatment, comprising: implanting into said subject in the area of said bone defect an osteogenic bone-compatible matrix, comprising a porous matrix infused with a peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof. The osteogenic bone-compatible matrix may be constructed of a material selected from the group consisting of natural cancellous bone, demineralized natural cancellous bone, collagen, calcium phosphate, hydroxyapatite, ceramic, polymer, metal, and metal alloy, and combinations thereof. The osteogenic bone-compatible matrix may be seeded with mesenchymal stem cells (MSCs).

In at least certain embodiments, the present disclosure is directed to a method of inducing human mesenchymal stem cells (hMSCs) to be converted into osteoblasts, comprising: exposing hMSCs, under conditions suitable for their growth, to at least one peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof, causing conversion of the hMSCs into osteoblasts. The hMSCs may be exposed to the peptide in vivo or in vitro.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the components, formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims. Applicants reserve the right to amend, add to, or replace the claims indicated herein below in this or subsequent patent applications.

1. A peptide conjugate, comprising at least one peptide linked to a carrier molecule or a carrier nanoparticle, the at least one peptide having an amino acid sequence selected from the group consisting of: SGTQDSMVGWNK (SEQ ID NO:3), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), TSKYLTMRPGPK (SEQ ID NO: 10), and variants thereof.

2. The peptide conjugate of claim 1, comprising the at least one peptide linked to a linker molecule.

3. The peptide conjugate of claim 1, comprising the at least one peptide displayed on a phage.

4. The peptide conjugate of claim 1, comprising the at least one peptide linked to a film.

5. The peptide conjugate of claim 1, wherein the at least one peptide has human mesenchymal stem cell (hMSC) stimulatory activity via binding to a bone morphogenic protein receptor (BMPR).

6. The peptide conjugate of claim 1, wherein the peptide variant has at least 75% identity to one of SEQ ID NO:2, SEQ ID NO:1, SEQ ID NO:3, SEQ ID NO:4, SEQ ID NO:5, and SEQ ID NO:6, SEQ ID NO:7, SEQ ID NO:8, SEQ ID NO:9, and SEQ ID NO: 10.

7. A peptide-implant complex, comprising at least one peptide linked to a surface of an implant, the at least one peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof.

8. The peptide-implant complex of claim 7, wherein the peptide-implant comprises a coating on the surface of the implant, the coating comprising the peptide.

9. The peptide-implant complex of claim 7, wherein the implant is a porous scaffold.

10. The peptide-implant complex of claim 7, wherein the implant comprises a material selected from the group consisting of natural cancellous bone, demineralized natural cancellous bone, collagen, calcium phosphate, hydroxyapatite, ceramic, polymer, metal, and metal alloy, and combinations thereof.

11. The peptide-bone implant complex of claim 10, wherein the metal alloy of the bone implant comprises titanium.

12. The peptide-bone implant complex of claim 7, seeded with mesenchymal stem cells (MSCs).

13. An osteogenic bone-compatible matrix, comprising a porous matrix infused with a peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof.

14. The osteogenic bone-compatible matrix of claim 13, wherein the osteogenic bone-compatible matrix is constructed of a material selected from the group consisting of natural cancellous bone, demineralized natural cancellous bone, collagen, calcium phosphate, hydroxyapatite, ceramic, polymer, metal, and metal alloy, and combinations thereof.

15. The osteogenic bone-compatible matrix of claim 13, seeded with mesenchymal stem cells (MSCs).

16. A method of treating a bone defect in a subject in need of such treatment, comprising: implanting into said subject in the area of said bone defect an osteogenic bone-compatible matrix, comprising a porous matrix infused with a peptide having an amino acid sequence selected from the group consisting of: VHVPLHRGAVSA (SEQ ID NO:2), TAKYLPMRPGPL (SEQ ID NO:1), SGTQDSMVGWNK (SEQ ID NO:3), RDYHPRDHTATW (SEQ ID NO:4), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof.

17. The method of claim 16, wherein the osteogenic bone-compatible matrix is constructed of a material selected from the group consisting of natural cancellous bone, demineralized natural cancellous bone, collagen, calcium phosphate, hydroxyapatite, ceramic, polymer, metal, and metal alloy, and combinations thereof.

18. The method of claim 16, wherein the osteogenic bone-compatible matrix is seeded with mesenchymal stem cells (MSCs).

19. A method of inducing human mesenchymal stem cells (hMSCs) to be converted into osteoblasts, comprising: exposing hMSCs, under conditions suitable for their growth, to at least one peptide having an amino acid sequence selected from the group consisting of: SGTQDSMVGWNK (SEQ ID NO:3), GNNPLHVHHDKR (SEQ ID NO:5), TAKSLPMRPGPL (SEQ ID NO:6), NAKYPTMRPGPK (SEQ ID NO:7), DYHDPSLPTLRK (SEQ ID NO:8), NAKYPTMRPGPL (SEQ ID NO:9), and TSKYLTMRPGPK (SEQ ID NO: 10), and variants thereof, causing conversion of the hMSCs into osteoblasts.

20. The method of claim 19, wherein the hMSCs are exposed to the peptide in vivo.

21. The method of claim 19, wherein the hMSCs are exposed to the peptide in vitro.


SEQUENCE LISTING (48 sequences)

SEQ ID NO: 1 - protein, 12 aa, Artificial sequence
  Completely synthesized in the lab - Ph.D.-12 Phage Display Peptide Library Kit (New England Biolabs)
TAKYLPMRPGPL

SEQ ID NO: 2 - protein, 12 aa, Artificial sequence
  Completely synthesized in the lab - Ph.D.-12 Phage Display Peptide Library Kit (New England Biolabs)
VHVPLHRGAVSA

SEQ ID NO: 3 - protein, 12 aa, Artificial sequence
  Completely synthesized in the lab - Ph.D.-12 Phage Display Peptide Library Kit (New England Biolabs)
SGTQDSMVGWNK

SEQ ID NO: 4 - protein, 12 aa, Artificial sequence
  Completely synthesized in the lab - Ph.D.-12 Phage Display Peptide Library Kit (New England Biolabs)
RDYHPRDHTATW

SEQ ID NO: 5 - protein, 12 aa, Artificial sequence
  Completely synthesized in the lab - Ph.D.-12 Phage Display Peptide Library Kit (New England Biolabs)
GNNPLHVHHDKR

SEQ ID NO: 6 - protein, 12 aa, Artificial sequence
  Completely synthesized in the lab - Ph.D.-12 Phage Display Peptide Library Kit (New England Biolabs)
TAKSLPMRPGPL

SEQ ID NO: 7 - protein, 12 aa, Artificial sequence
  Completely synthesized in the lab - Ph.D.-12 Phage Display Peptide Library Kit (New England Biolabs)
NAKYPTMRPGPK

SEQ ID NO: 8 - protein, 12 aa, Artificial sequence
  Completely synthesized in the lab - Ph.D.-12 Phage Display Peptide Library Kit (New England Biolabs)
DYHDPSLPTLRK

SEQ ID NO: 9 - protein, 12 aa, Artificial sequence
  Completely synthesized in the lab - Ph.D.-12 Phage Display Peptide Library Kit (New England Biolabs)
NAKYPTMRPGPL

SEQ ID NO: 10 - protein, 12 aa, Artificial sequence
  Completely synthesized in the lab - Ph.D.-12 Phage Display Peptide Library Kit (New England Biolabs)
TSKYLTMRPGPK

SEQ ID NO: 11 - protein, 12 aa, Artificial sequence
  Completely synthesized in the lab - Ph.D.-12 Phage Display Peptide Library Kit (New England Biolabs)
DYHDPSLPTVRK

SEQ ID NO: 12 - protein, 12 aa, Artificial sequence
  Completely synthesized in the lab - Ph.D.-12 Phage Display Peptide Library Kit (New England Biolabs)
GNHPPHVHHDQH

SEQ ID NO: 13 - protein, 12 aa, Artificial sequence
  Completely synthesized in the lab - Ph.D.-12 Phage Display Peptide Library Kit (New England Biolabs)
GNKSLHVHPGKR

SEQ ID NO: 14 - protein, 12 aa, Artificial sequence
  Completely synthesized in the lab - Ph.D.-12 Phage Display Peptide Library Kit (New England Biolabs)
NMKVATLHEYYN

SEQ ID NO: 15 - protein, 12 aa, Artificial sequence
  Completely synthesized in the lab - Ph.D.-12 Phage Display Peptide Library Kit (New England Biolabs)
NMKVATLNEYYN

SEQ ID NO: 16 - protein, 12 aa, Artificial sequence
  Completely synthesized in the lab - Ph.D.-12 Phage Display Peptide Library Kit (New England Biolabs)
GNNSLHVHPDQR

SEQ ID NO: 17 - protein, 12 aa, Artificial sequence
  Completely synthesized in the lab - Ph.D.-12 Phage Display Peptide Library Kit (New England Biolabs)
GAKSLPMRPGPL

SEQ ID NO: 18 - protein, 12 aa, Artificial sequence
  Completely synthesized in the lab - Ph.D.-12 Phage Display Peptide Library Kit (New England Biolabs)
GNKSLHVHHDKR

SEQ ID NO: 19 - protein, 12 aa, Artificial sequence
  Completely synthesized in the lab - Ph.D.-12 Phage Display Peptide Library Kit (New England Biolabs)
DYHYPSLPPVPK

SEQ ID NO: 20 - protein, 12 aa, Artificial sequence
  Completely synthesized in the lab - Ph.D.-12 Phage Display Peptide Library Kit (New England Biolabs)
NDKTGKAISRNQ

SEQ ID NO: 21 - protein, 12 aa, Artificial sequence
  Completely synthesized in the lab - Ph.D.-12 Phage Display Peptide Library Kit (New England Biolabs)
DYHDPSLLPVRK

SEQ ID NO: 22 - protein, 12 aa, Artificial sequence
  Completely synthesized in the lab - Ph.D.-12 Phage Display Peptide Library Kit (New England Biolabs)
  misc_feature (4)..(4): Xaa can be any naturally occurring amino acid
DYHXPSLLPVRK

SEQ ID NO: 23 - protein, 12 aa, Artificial sequence
  Completely synthesized in the lab - Ph.D.-12 Phage Display Peptide Library Kit (New England Biolabs)
DWHDPSLLAVRK

SEQ ID NO: 24 - protein, 12 aa, Artificial sequence
  Completely synthesized in the lab - Ph.D.-12 Phage Display Peptide Library Kit (New England Biolabs)
GYKSPHLLPVPR

SEQ ID NO: 25 - protein, 12 aa, Artificial sequence
  Completely synthesized in the lab - Ph.D.-12 Phage Display Peptide Library Kit (New England Biolabs)
GKKSLPMRHDTR

SEQ ID NO: 26 - protein, 12 aa, Artificial sequence
  Completely synthesized in the lab - Ph.D.-12 Phage Display Peptide Library Kit (New England Biolabs)
SGTQDSMVGWNK

SEQ ID NO: 27 - protein, 12 aa, Artificial sequence
  Completely synthesized in the lab - Ph.D.-12 Phage Display Peptide Library Kit (New England Biolabs)
SPWLDMVELRRP

SEQ ID NO: 28 - protein, 12 aa, Artificial sequence
  Completely synthesized in the lab - Ph.D.-12 Phage Display Peptide Library Kit (New England Biolabs)
RHYQRRGEIFTG

SEQ ID NO: 29 - protein, 12 aa, Artificial sequence
  Completely synthesized in the lab - Ph.D.-12 Phage Display Peptide Library Kit (New England Biolabs)
HLEALSDLVNRN

SEQ ID NO: 30 - protein, 12 aa, Artificial sequence
  Completely synthesized in the lab - Ph.D.-12 Phage Display Peptide Library Kit (New England Biolabs)
ADRFCDMSQCAP

SEQ ID NO: 31 - protein, 12 aa, Artificial sequence
  Completely synthesized in the lab - Ph.D.-12 Phage Display Peptide Library Kit (New England Biolabs)
SFNWRYIEMSRW

SEQ ID NO: 32 - protein, 12 aa, Artificial sequence
  Completely synthesized in the lab - Ph.D.-12 Phage Display Peptide Library Kit (New England Biolabs)
SGIQDSMVGWNK

SEQ ID NO: 33 - protein, 12 aa, Artificial sequence
  Completely synthesized in the lab - Ph.D.-12 Phage Display Peptide Library Kit (New England Biolabs)
TAKYLPMRPGPL

SEQ ID NO: 34 - protein, 12 aa, Artificial sequence
  Completely synthesized in the lab - Ph.D.-12 Phage Display Peptide Library Kit (New England Biolabs)
RDYQPREHGGNR

SEQ ID NO: 35 - protein, 12 aa, Artificial sequence
  Completely synthesized in the lab - Ph.D.-12 Phage Display Peptide Library Kit (New England Biolabs)
  misc_feature (7)..(7): Xaa can be any naturally occurring amino acid
  misc_feature (9)..(9): Xaa can be any naturally occurring amino acid
RDFHPRXHXATW

SEQ ID NO: 36 - protein, 12 aa, Artificial sequence
  Completely synthesized in the lab - Ph.D.-12 Phage Display Peptide Library Kit (New England Biolabs)
DYHDPSLPTLRK

SEQ ID NO: 37 - protein, 12 aa, Artificial sequence
  Completely synthesized in the lab - Ph.D.-12 Phage Display Peptide Library Kit (New England Biolabs)
FDDVYWRWTYTA

SEQ ID NO: 38 - protein, 12 aa, Artificial sequence
  Completely synthesized in the lab - Ph.D.-12 Phage Display Peptide Library Kit (New England Biolabs)
QVNGLGERSQQM

SEQ ID NO: 39 - protein, 12 aa, Artificial sequence
  Completely synthesized in the lab - Ph.D.-12 Phage Display Peptide Library Kit (New England Biolabs)
SGLSSSWSYCYL

SEQ ID NO: 40 - DNA, 36 bp, Artificial Sequence
  Completely synthesized in the lab.
actgcgaagtatctgcctatgcgtcctgggccgctt

SEQ ID NO: 41 - DNA, 36 bp, Artificial Sequence
  Completely synthesized in the lab.
gttcatgttccgttgcataggggtgcggtttcggcg

SEQ ID NO: 42 - DNA, 36 bp, Artificial Sequence
  Completely synthesized in the lab.
tctggtacttaggattctatggttggttggaataag

SEQ ID NO: 43 - DNA, 36 bp, Artificial Sequence
  Completely synthesized in the lab.
cgggattatcatcctcgtgatcatactgctacttgg

SEQ ID NO: 44 - DNA, 61 bp, Artificial Sequence
  Completely synthesized in the lab.
atccatggcggttcatgttccgttgcataggggtgcggtttcggcggatcccgcaaaagcg

SEQ ID NO: 45 - DNA, 61 bp, Artificial Sequence
  Completely synthesized in the lab.
atccatggcgtctggtacttaggattctatggttggttggaataaggatcccgcaaaagcg

SEQ ID NO: 46 - DNA, 61 bp, Artificial Sequence
  Completely synthesized in the lab.
atccatggcgcgggattatcatcctcgtgatcatactgctacttgggatcccgcaaaagcg

SEQ ID NO: 47 - DNA, 61 bp, Artificial Sequence
  Completely synthesized in the lab.
atccatggcgactgcgaagtatctgcctatgcgtcctgggccgcttgatcccgcaaaagcg

SEQ ID NO: 48 - DNA, 27 bp, Artificial Sequence
  Completely synthesized in the lab.
gcaagcttttatcagcttgctttcgag